(19) United States
(12) Patent Application Publication
(10) Pub. No.: US 2026/0096717 A1
(43) Pub. Date: Apr. 9, 2026

(54) INSERTION STATE DETERMINATION SYSTEM, INSERTION STATE DETERMINATION METHOD, AND RECORDING MEDIUM

(71) Applicant: Evident Inspection Technologies Japan Corporation, Nagano (JP)

(72) Inventor: Yoshiyuki SHIGEHISA, Fuchu-shi (JP)

(73) Assignee: Evident Inspection Technologies Japan Corporation, Nagano (JP)

(21) Appl. No.: 19/416,151

(22) Filed: Dec. 11, 2025

Related U.S. Application Data
(62) Division of application No. 18/138,967, filed on Apr. 25, 2023.

(30) Foreign Application Priority Data
Jun. 27, 2022 (JP) 2022-102793

(51) Int. Cl. A61B 1/005; A61B 1/00

(52) U.S. Cl. CPC A61B 1/0051; A61B 1/00147

(57) ABSTRACT

An insertion state determination system includes a sensor unit including a first sensor and the system includes a second sensor and a processor. The first sensor is configured to determine a first rotation amount indicating a rotation amount of an elongated insertion unit of an endoscope device around a center axis of the insertion unit. A hole through which the insertion unit passes is formed in the sensor unit. The second sensor is disposed in the sensor unit or an object fixed to the sensor unit and is configured to determine a second rotation amount indicating a rotation amount of the sensor unit around the center axis when the insertion unit is inserted into the subject. The processor is configured to calculate a corrected rotation amount by correcting the first rotation amount based on the second rotation amount.

### BACKGROUND OF THE INVENTION

### Field of the Invention

The present invention relates to an insertion state determination system, an insertion state determination method, and a recording medium.

This application is a divisional application of U.S. patent application Ser. No. 18/138,967, filed on Apr. 25, 2023, which claims priority to Japanese Patent Application No. 2022-102793, filed on Jun. 27, 2022, the content of each of which is incorporated herein by reference.

### Description of Related Art

Industrial endoscope devices have been used for inspection of internal abnormalities, corrosion, and the like of boilers, pipes, aircraft engines, and the like. An endoscope device includes an insertion unit used for acquiring an image. A user inserts the insertion unit into a subject and acquires an image of an inspection portion in the subject. The user observes the image and inspects the inspection portion. When an abnormal part is found in the inspection portion, the user measures the size of the part.

A user performs an insertion operation in order to insert the insertion unit into a subject. The insertion operation includes an operation of pushing or pulling the insertion unit, an operation of rotating the insertion unit, an operation of adjusting the posture of the insertion unit, and an operation of bending a distal end portion of the insertion unit. These operations are combined in accordance with the internal structure of the subject.

A user observes an image acquired by the insertion unit and performs the insertion operation. In a case in which the proficiency of the user is poor, the distal end of the insertion unit may touch a wall in the subject and the insertion unit may stop advancing. Alternatively, there is a case in which the insertion unit tends to easily bend in a certain direction. Therefore, even when an unskilled user pushes and inserts the insertion unit into the subject, the distal end portion may bend and the insertion unit may stop advancing.

A technique disclosed in Japanese Unexamined Patent Application, First Publication No. 2014-113352 provides a navigation function for outputting insertion assistance information in accordance with the state of an insertion unit. The technique uses a sensor that determines a relative rotation amount of the insertion unit to a holding unit, a sensor that determines a positional relationship between the insertion unit and a subject, and a sensor that determines a bending state of the insertion unit. The positional relationship indicates the length of the insertion unit inserted into the subject, a relative rotation amount of the insertion unit to the subject, and the direction of the insertion unit with respect to the subject. The technique processes information determined by these sensors and generates insertion assistance information.

An unskilled user can perform an operation required for inserting the insertion unit into the subject by referring to the insertion assistance information provided by the above-described navigation function. Therefore, work efficiency and inspection quality are improved.

In addition, the following effects are expected by using information determined by each sensor. In an inspection using an endoscope device, an inspection result is recorded. The inspection result includes a still image of an inspection portion and a measurement result. In addition, the inspection result and the state of the insertion unit are associated with each other, and the inspection result and the state of the insertion unit are recorded. A user can confirm that the inspection has been performed in accordance with an inspection plan by referring to the inspection result and the state of the insertion unit. In addition, when an inspection is performed next, the user can easily locate an inspection portion that should be paid attention to.

### SUMMARY OF THE INVENTION

According to a first aspect of the present invention, an insertion state determination system includes a sensor unit including a first sensor and the system includes a second sensor and a processor. The first sensor is configured to determine a first rotation amount when an elongated insertion unit of an endoscope device is inserted into a subject. The first rotation amount indicates a rotation amount of the insertion unit around a center axis of the insertion unit. A hole through which the insertion unit passes is formed in the sensor unit. The second sensor is disposed in the sensor unit or an object fixed to the sensor unit and is configured to determine a second rotation amount indicating a rotation amount of the sensor unit around the center axis when the insertion unit is inserted into the subject. The processor is configured to acquire the first rotation amount and the second rotation amount, and calculate a corrected rotation amount by correcting the first rotation amount based on the second rotation amount.

According to a second aspect of the present invention, in the first aspect, the first sensor may be configured to determine a moving amount indicating an amount by which the insertion unit moves in a longitudinal direction of the insertion unit when the insertion unit is inserted into the subject.

According to a third aspect of the present invention, in the second aspect, the processor may be configured to record insertion state information including the corrected rotation amount and the moving amount associated with each other on a recording medium.

According to a fourth aspect of the present invention, in the third aspect, the processor may be configured to record insertion state information including the second rotation amount and the moving amount associated with each other on a recording medium.

According to a fifth aspect of the present invention, in the third aspect, the second sensor may be configured to determine a posture of the sensor unit. The insertion state information may further include posture information that is associated with the moving amount and indicates the posture.

According to a sixth aspect of the present invention, in the fourth aspect, the second sensor may be configured to determine a posture of the sensor unit. The insertion state information may further include posture information that is associated with the moving amount and indicates the posture.

According to a seventh aspect of the present invention, in the third aspect, the insertion unit may include a third sensor that is disposed in a distal end portion including a distal end of the insertion unit and is configured to determine a posture of the distal end portion. The insertion state information may further include posture information that is associated with the moving amount and indicates the posture.

According to an eighth aspect of the present invention, in the fourth aspect, the insertion unit may include a third sensor that is disposed in a distal end portion including a distal end of the insertion unit and is configured to determine a posture of the distal end portion. The insertion state information may further include posture information that is associated with the moving amount and indicates the posture.

According to a ninth aspect of the present invention, in the third aspect, a distal end portion including a distal end of the insertion unit may be bendable inside the subject based on a bending instruction input through an input device that accepts an operation performed by a user. The insertion state information may further include a bending amount that is associated with the moving amount and indicates an amount by which the distal end portion has bent.

According to a tenth aspect of the present invention, in the fourth aspect, a distal end portion including a distal end of the insertion unit may be bendable inside the subject based on a bending instruction input through an input device that accepts an operation performed by a user. The insertion state information may further include a bending amount that is associated with the moving amount and indicates an amount by which the distal end portion has bent.

According to an eleventh aspect of the present invention, in the third aspect, the processor may be configured to generate operation information indicating an operation required for inserting the insertion unit into the subject by using the corrected rotation amount calculated in real time and the corrected rotation amount included in the insertion state information recorded on the recording medium.

According to a twelfth aspect of the present invention, in the fourth aspect, the processor may be configured to generate operation information indicating an operation required for inserting the insertion unit into the subject by using the corrected rotation amount calculated in real time and the corrected rotation amount included in the insertion state information recorded on the recording medium.

According to a thirteenth aspect of the present invention, in the eleventh aspect, the processor may be configured to calculate a difference between the corrected rotation amount calculated in real time and the corrected rotation amount included in the insertion state information recorded on the recording medium and generate the operation information by using the difference.

According to a fourteenth aspect of the present invention, in the twelfth aspect, the processor may be configured to calculate a difference between the corrected rotation amount calculated in real time and the corrected rotation amount included in the insertion state information recorded on the recording medium and generate the operation information by using the difference.

According to a fifteenth aspect of the present invention, in the first aspect, the insertion unit may include a third sensor that is disposed in a distal end portion including a distal end of the insertion unit and is configured to determine a third rotation amount indicating a rotation amount of the insertion unit around a center axis of the insertion unit. The processor may be configured to reset a relative rotation amount of the insertion unit to the sensor unit by using the second rotation amount and the third rotation amount.

According to a sixteenth aspect of the present invention, in the first aspect, a distal end portion including a distal end of the insertion unit may be bendable inside the subject based on a bending instruction input through an input device that accepts an operation performed by a user. The second sensor may be disposed in the input device.

According to a seventeenth aspect of the present invention, in the sixteenth aspect, the input device may be attachable to and detachable from the sensor unit. When the input device is attached to the sensor unit, the second sensor may be configured to determine the second rotation amount.

According to an eighteenth aspect of the present invention, in the first aspect, the processor may be configured to calculate the corrected rotation amount by performing addition or subtraction using the first rotation amount and the second rotation amount.

According to a nineteenth aspect of the present invention, an insertion state determination method is executed by a processor. The method includes acquiring a first rotation amount when an elongated insertion unit of an endoscope device is inserted into a subject. The first rotation amount indicates a rotation amount of the insertion unit around a center axis of the insertion unit and is determined by a first sensor disposed in a sensor unit in which a hole through which the insertion unit passes is formed. The method includes acquiring a second rotation amount when the insertion unit is inserted into the subject. The second rotation amount indicates a rotation amount of the sensor unit around the center axis and is determined by a second sensor disposed in the sensor unit or an object fixed to the sensor unit. The method includes calculating a corrected rotation amount by correcting the first rotation amount based on the second rotation amount.

According to a twentieth aspect of the present invention, a non-transitory computer-readable recording medium stores a program causing a computer to execute processing. The computer acquires a first rotation amount when an elongated insertion unit of an endoscope device is inserted into a subject. The first rotation amount indicates a rotation amount of the insertion unit around a center axis of the insertion unit and is determined by a first sensor disposed in a sensor unit in which a hole through which the insertion unit passes is formed. The computer acquires a second rotation amount when the insertion unit is inserted into the subject. The second rotation amount indicates a rotation amount of the sensor unit around the center axis and is determined by a second sensor disposed in the sensor unit or an object fixed to the sensor unit. The computer calculates a corrected rotation amount by correcting the first rotation amount based on the second rotation amount.

### BRIEF DESCRIPTION OF THE DRAWINGS

FIG. 1 is a perspective view showing an entire configuration of an endoscope device according to a first embodiment of the present invention.

FIG. 2 is a diagram showing a state of a sensor unit in the first embodiment of the present invention.

FIG. 3 is a diagram showing a state of the sensor unit in the first embodiment of the present invention.

FIG. 4 is a block diagram showing an internal configuration of the endoscope device according to the first embodiment of the present invention.

FIG. 5 is a cross-sectional view showing a configuration of the sensor unit in the first embodiment of the present invention.

FIG. 6 is a diagram showing a configuration of an optical sensor in the first embodiment of the present invention.

FIG. 7 is a cross-sectional view showing a configuration of the sensor unit in the first embodiment of the present invention.

FIG. 8 is a cross-sectional view showing a configuration of an operation unit and the sensor unit in the first embodiment of the present invention.

FIG. 9 is a graph showing an example of a change of states of an insertion unit and the sensor unit in the first embodiment of the present invention.

FIG. 10 is a flow chart showing an entire procedure of an insertion operation in the first embodiment of the present invention.

FIG. 11 is a flow chart showing a procedure of state-recording processing in the first embodiment of the present invention.

FIG. 12 is a cross-sectional view showing a positional relationship between the insertion unit and the sensor unit in the first embodiment of the present invention.

FIG. 13 is a cross-sectional view showing a positional relationship between a subject and the insertion unit in the first embodiment of the present invention.

FIG. 14 is a flow chart showing a procedure of history-recording processing in the first embodiment of the present invention.

FIG. 15 is a graph showing an example of a change of states of the insertion unit and the sensor unit in the first embodiment of the present invention.

FIG. 16 is a flow chart showing a procedure of equipment-setting processing in the first embodiment of the present invention.

FIG. 17 is a diagram showing information displayed on a display unit in the first embodiment of the present invention.

FIG. 18 is a flow chart showing a procedure of insertion assistance processing in the first embodiment of the present invention.

FIG. 19 is a diagram showing information displayed on the display unit in the first embodiment of the present invention.

FIG. 20 is a flow chart showing a procedure of state-recording processing in a second embodiment of the present invention.

FIG. 21 is a cross-sectional view showing a positional relationship between an insertion unit and a sensor unit in the second embodiment of the present invention.

FIG. 22 is a cross-sectional view showing a positional relationship between the insertion unit and the sensor unit in the second embodiment of the present invention.

FIG. 23 is a flow chart showing a procedure of equipment-setting processing in the second embodiment of the present invention.

FIG. 24 is a diagram showing information displayed on a display unit in the second embodiment of the present invention.

FIG. 25 is a diagram showing information displayed on the display unit in the second embodiment of the present invention.

FIG. 26 is a cross-sectional view showing a configuration of an operation unit and a sensor unit in a third embodiment of the present invention.

FIG. 27 is a cross-sectional view showing a configuration of an operation unit and a sensor unit in a fourth embodiment of the present invention.

FIG. 28 is a block diagram showing an internal configuration of an endoscope device according to a fifth embodiment of the present invention.

### DETAILED DESCRIPTION OF THE INVENTION

Hereinafter, embodiments of the present invention will be described with reference to the drawings.

### First Embodiment

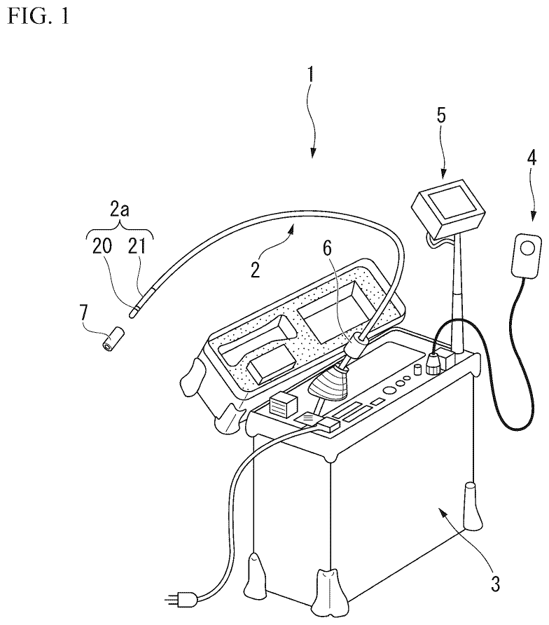

FIG. 1 shows an external appearance of an endoscope device 1 (insertion state determination system) according to a first embodiment of the present invention. The endoscope device 1 shown in FIG. 1 includes an insertion unit 2, a main body unit 3, an operation unit 4, a display unit 5, and a sensor unit 6.

The insertion unit 2 is to be inserted into the inside of a subject. A user (inspector) performs an insertion operation and inserts the insertion unit 2 into the subject. The insertion unit 2 has an elongated tubular shape. The insertion unit 2 includes a distal end portion 2a. The distal end portion 2a includes an imaging portion 20 and a bending portion 21. The imaging portion 20 includes the distal end of the insertion unit 2 and is formed of a rigid material. An optical adaptor 7 is mounted on the imaging portion 20. The bending portion 21 is disposed on the base end side of the imaging portion 20. The bending portion 21 is bendable in a predetermined direction. The insertion unit 2 converts an optical image of the subject into an imaging signal and outputs the imaging signal to the main body unit 3.

The main body unit 3 is a control device including a housing unit that houses the insertion unit 2. The operation unit 4 accepts an operation for the endoscope device 1 from a user. The display unit 5 includes a display screen and displays an image of a subject acquired by the insertion unit 2 on the display screen.

The operation unit 4 is a user interface (input device). For example, the operation unit 4 is at least one of a button, a switch, a key, a mouse, a joystick, a touch pad, a track ball, and a touch panel. A user bends the bending portion 21 by performing a bending operation using the operation unit 4. Alternatively, the user controls the state of illumination by operating the operation unit 4. In addition, the user inputs information used for setting the state of the endoscope device 1 into the endoscope device 1 by operating the operation unit 4. An input device including the operation unit 4 may be connected to the main body unit 3 by using wired or wireless connection.

The display unit 5 is a monitor (display) such as a liquid crystal display (LCD). The display unit 5 may be a touch panel. In such a case, the operation unit 4 and the display unit 5 are integrated. A user touches the screen of the display unit 5 by using a part of the body or a tool. For example, the part of the body is the finger. The display unit 5 may be connected to the main body unit 3 by using wired or wireless connection. An information terminal such as a tablet, a smartphone, or a personal computer may be used as a terminal including the operation unit 4 and the display unit 5.

A tubular hole through which the insertion unit 2 passes is formed in the sensor unit 6. The insertion unit 2 is capable of moving in the sensor unit 6. The sensor unit 6 determines an insertion length indicating the length of a portion of the insertion unit 2 inserted into a space in a subject. The insertion length corresponds to the position of the imaging portion 20. In addition, the sensor unit 6 determines a rotation amount of the insertion unit 2 around a center axis of the insertion unit 2.

A user performs the bending operation and the insertion operation while viewing an image displayed on the display unit 5. When the insertion unit 2 is inserted into a subject, the endoscope device 1 assists the insertion operation. The user locates an inspection portion and disposes the imaging portion 20 so that the inspection portion can be seen in an image in an appropriate state. Thereafter, the user performs an inspection of the subject. For example, the user determines the degree of deterioration of the subject in the inspection.

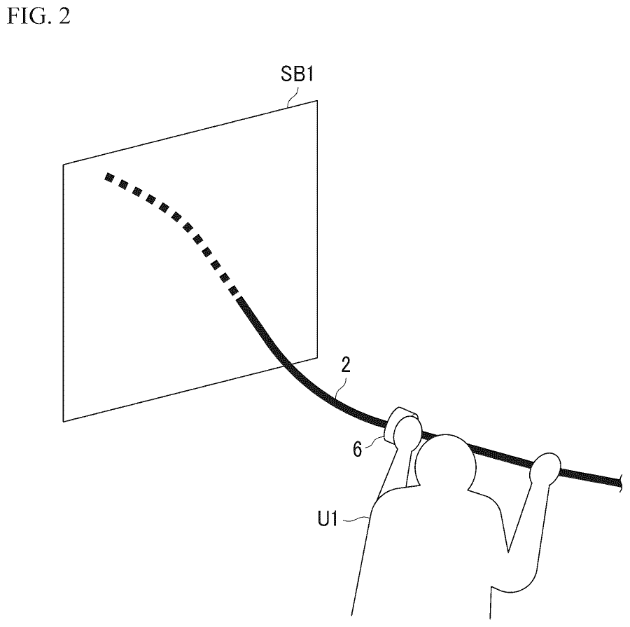

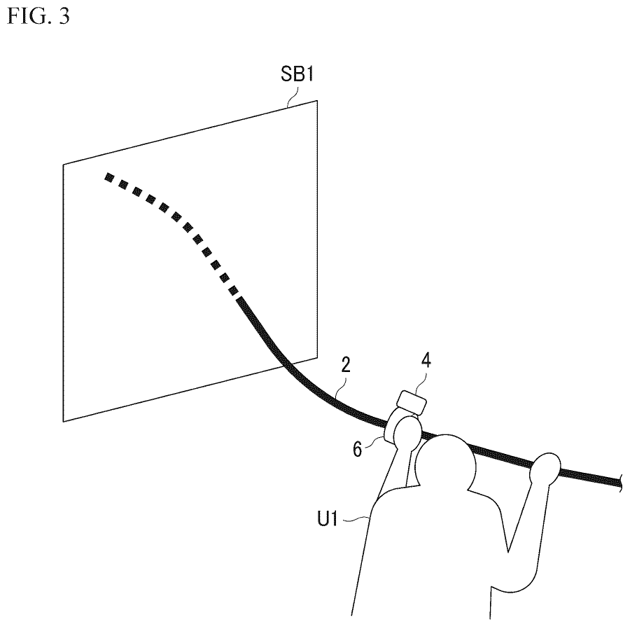

FIG. 2 and FIG. 3 show the state of the sensor unit 6 in an inspection. FIG. 2 shows a first example. FIG. 3 shows a second example. A user U1 inserts the insertion unit 2 into a subject SB1.

In the first example shown in FIG. 2, the user U1 holds the sensor unit 6 with the left hand and holds the insertion unit 2 with the right hand. The user U1 may hold the sensor unit 6 with the right hand and may hold the insertion unit 2 with the left hand. Since the sensor unit 6 is not fixed to the subject SB1, the sensor unit 6 can be disposed not only near the subject SB1 but also at any position.

In the second example shown in FIG. 3, the operation unit 4 and the sensor unit 6 are integrated. The user U1 holds one or both of the operation unit 4 and the sensor unit 6 with the left hand and holds the insertion unit 2 with the right hand. The user U1 may hold one or both of the operation unit 4 and the sensor unit 6 with the right hand and may hold the insertion unit 2 with the left hand. The user U1 can operate the operation unit 4 and can hold the sensor unit 6 at the same time.

In a case in which the shape of a subject is known in advance such as a case in which the subject is an aircraft engine, the sensor unit 6 may be configured to be fixed to the surface of the subject. An auxiliary component may be used for fixing the sensor unit 6. For example, the auxiliary component may be fixed to the surface of the subject, and the sensor unit 6 may be fixed to the auxiliary component.

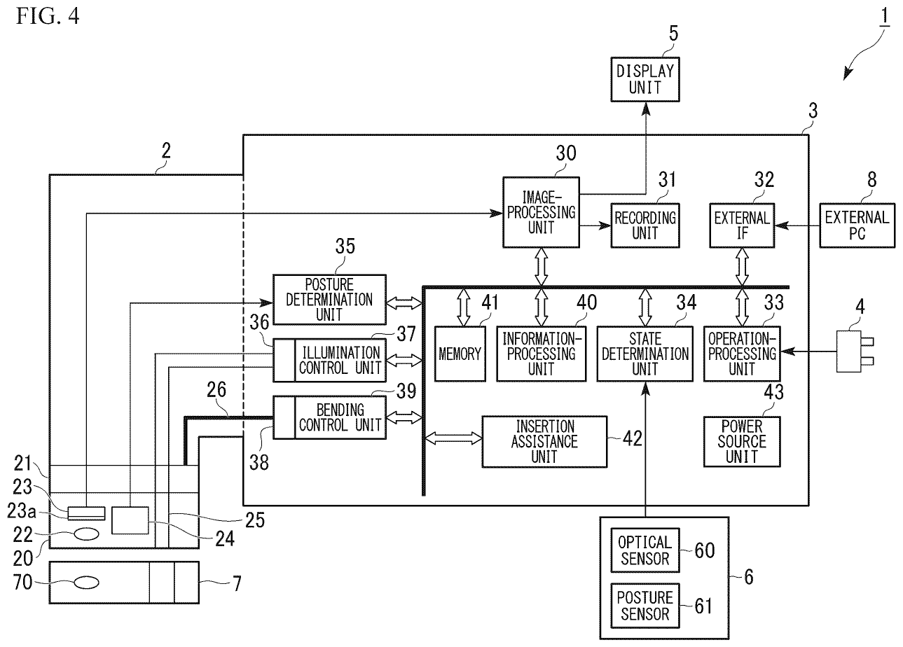

FIG. 4 shows an internal configuration of the endoscope device 1. The imaging portion 20 of the insertion unit 2 includes a lens 22, an imaging device 23, and a posture sensor 24.

The main body unit 3 includes an image-processing unit 30, a recording unit 31, an external interface (IF) 32, an operation-processing unit 33, a state determination unit 34, a posture determination unit 35, a light source 36, an illumination control unit 37, a motor 38, a bending control unit 39, an information-processing unit 40, a memory 41, an insertion assistance unit 42, and a power source unit 43.

The optical adaptor 7 includes a lens 70. Light incident on the lens 70 passes through the lens 70 and is incident on the lens 22. The lens 70 and the lens 22 constitute an observation optical system. The light incident on the lens 22 passes through the lens 22 and is incident on the imaging device 23. The imaging device 23 is an image sensor such as a CCD sensor or a CMOS sensor. The imaging device 23 includes an imaging surface 23a on which the light passing through the lens 22 is incident. The imaging device 23 converts the light incident on the imaging surface 23a into an imaging signal.

The imaging signal generated by the imaging device 23 includes an image of a subject. Accordingly, the imaging device 23 acquires an optical image of the subject and generates an image of the subject. The image generated by the imaging device 23 is output to the main body unit 3.

The posture sensor 24 includes at least one of a 3-axis acceleration sensor, a 3-axis gyro sensor, and a 3-axis geomagnetic sensor. The posture sensor 24 determines a value related to the posture of the imaging portion 20 and outputs the determined value to the main body unit 3. The value indicates at least one of an acceleration, an angular velocity, and a geomagnetic field.

The posture sensor 24 may include only one of an acceleration sensor, a gyro sensor, and a geomagnetic sensor. The posture sensor 24 may include any two or three of the acceleration sensor, the gyro sensor, and the geomagnetic sensor. For example, the posture sensor 24 may include the acceleration sensor and the gyro sensor. Alternatively, the posture sensor 24 may include the acceleration sensor, the gyro sensor, and the geomagnetic sensor. The posture sensor 24 may be unnecessary.

The image-processing unit 30 processes the imaging signal output from the imaging device 23, thus processing an image of a subject. For example, the image-processing unit 30 executes processing such as noise elimination, brightness adjustment, and color adjustment in order to enhance the quality of the image. The image-processing unit 30 may execute localization such as visual simultaneous localization and mapping (Visual SLAM) and may calculate a position and a posture of the imaging portion 20. Furthermore, the image-processing unit 30 superimposes insertion assistance information generated by the insertion assistance unit 42 on the image of the subject.

The image processed by the image-processing unit 30 is output to the display unit 5 or the recording unit 31. The display unit 5 displays the image processed by the image-processing unit 30. The recording unit 31 includes a recording medium and records the image processed by the image-processing unit 30 on the recording medium.

The external IF 32 is connected to an external PC 8. The external PC 8 is a versatile personal computer. An information terminal such as a tablet or a smartphone may be used instead of the external PC 8. The external IF 32 may be connected to a server on a network (cloud). The external IF 32 may be connected to a recording medium such as a memory card.

The operation-processing unit 33 is connected to the operation unit 4. The operation unit 4 outputs information in accordance with an operation performed by a user. The operation-processing unit 33 sets the state of the endoscope device 1 in accordance with the information output from the operation unit 4.

The sensor unit 6 is a cabinet (case) that houses an optical sensor 60 and a posture sensor 61. The optical sensor 60 determines a value related to a moving amount of the insertion unit 2 in the longitudinal direction of the insertion unit 2. By doing this, the optical sensor 60 determines a value related to the insertion length of the insertion unit 2. In addition, the optical sensor 60 determines a value related to a rotation amount of the insertion unit 2 around the center axis of the insertion unit 2. The optical sensor 60 can determine a value related to a moving amount of the insertion unit 2 and a value related to a rotation amount of the insertion unit 2 without touching the insertion unit 2.

The posture sensor 61 determines a value related to a rotation amount of the sensor unit 6 around the center axis of the insertion unit 2. In addition, the posture sensor 61 determines a value related to the posture of the sensor unit 6. The sensor unit 6 outputs the determined values to the state determination unit 34. Details of the sensor unit 6 will be described later.

The state determination unit 34 calculates an insertion length and a rotation amount of the insertion unit 2 based on the values output from the sensor unit 6. In addition, the state determination unit 34 calculates a posture of the sensor unit 6 based on the value output from the sensor unit 6 and generates posture information indicating the posture. Since the insertion unit 2 passes through the hole formed in the sensor unit 6, the posture information of the sensor unit 6 indicates the posture of the insertion unit 2 in the hole. For example, the posture information indicates the angle of the center axis of the insertion unit 2.

The posture determination unit 35 determines the posture of the imaging portion 20 based on the value output from the posture sensor 24 and generates posture information indicating the posture.

The light source 36 is a light-emitting diode (LED) or the like and generates illumination light. The illumination light is lead to the imaging portion 20 via a light guide 25 disposed in the insertion unit 2. The illumination light is emitted from the imaging portion 20 to a subject. The illumination control unit 37 controls the light source 36 based on the information output from the operation unit 4, thus setting turning-on and turning-off of illumination and the intensity of the illumination.

The motor 38 is connected to a plurality of angle wires 26. The plurality of angle wires 26 are disposed in the insertion unit 2 and are connected to the bending portion 21. The motor 38 pulls the plurality of angle wires 26, thus bending the bending portion 21. The bending control unit 39 controls the motor 38 based on the information output from the operation unit 4, thus controlling the angle of the bending portion 21. In other words, the bending control unit 39 controls the posture of the imaging portion 20.

The information-processing unit 40 processes information generated by the state determination unit 34. Specifically, the information-processing unit 40 associates the insertion length of the insertion unit 2, the rotation amount of the insertion unit 2, and the posture information of the sensor unit 6 with each other and generates insertion state information. The insertion state information may include the posture information of the imaging portion 20. The insertion state information may include a bending amount indicating the amount by which the bending portion 21 has bent. The information-processing unit 40 records the insertion state information on the memory 41.

The memory 41 stores the insertion state information including the insertion length, the rotation amount, and the posture information. The memory 41 is a nonvolatile recording medium. For example, the memory 41 is at least one of a static random-access memory (SRAM), an erasable programmable read-only memory (EPROM), an electrically erasable programmable read-only memory (EEPROM), and a flash memory.

The insertion assistance unit 42 generates insertion assistance information. The insertion assistance information includes information used for assisting the insertion operation. The insertion assistance unit 42 outputs the insertion assistance information to the display unit 5 via the image-processing unit 30. By doing this, the insertion assistance unit 42 displays the insertion assistance information on the display unit 5.

The power source unit 43 supplies each unit of the endoscope device 1 with driving power.

At least one of the image-processing unit 30, the operation-processing unit 33, the state determination unit 34, the posture determination unit 35, the illumination control unit 37, the bending control unit 39, the information-processing unit 40, and the insertion assistance unit 42 may be constituted by at least one of a processor and a logic circuit. For example, the processor is at least one of a central processing unit (CPU), a digital signal processor (DSP), and a graphics-processing unit (GPU). For example, the logic circuit is at least one of an application-specific integrated circuit (ASIC) and a field-programmable gate array (FPGA). The image-processing unit 30 and the like may include one or a plurality of processors. The image-processing unit 30 and the like may include one or a plurality of logic circuits.

A computer of the endoscope device 1 may read a program and execute the read program. The program includes commands defining the operations of the image-processing unit 30 and the like. In other words, the functions of the image-processing unit 30 and the like may be realized by software.

The program described above, for example, may be provided by using a “computer-readable recording medium” such as a flash memory. The program may be transmitted from the computer storing the program to the endoscope device 1 through a transmission medium or transmission waves in a transmission medium. The “transmission medium” transmitting the program is a medium having a function of transmitting information. The medium having the function of transmitting information includes a network (communication network) such as the Internet and a communication circuit line (communication line) such as a telephone line. The program described above may realize some of the functions described above. In addition, the program described above may be a differential file (differential program). The functions described above may be realized by a combination of a program that has already been recorded in a computer and a differential program.

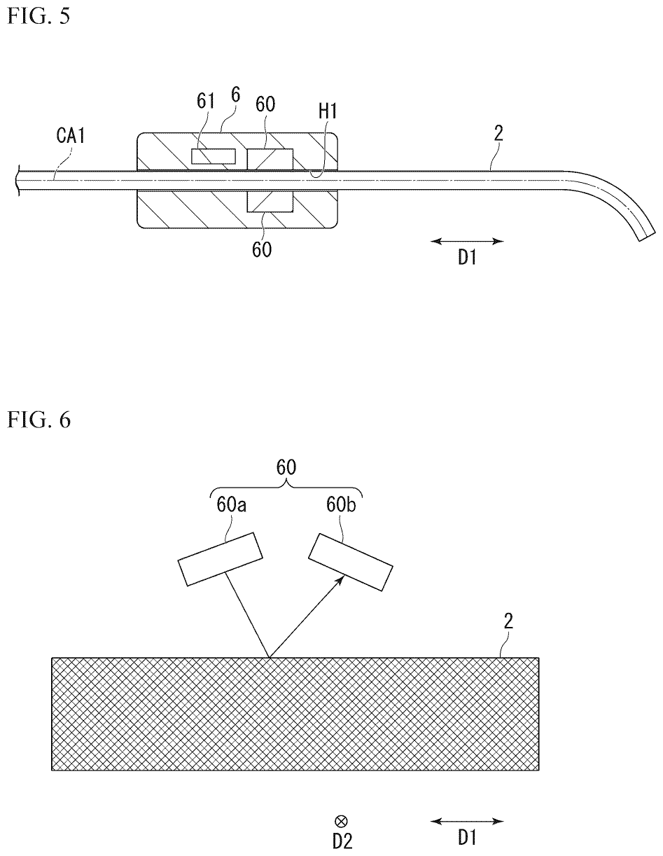

FIG. 5 shows a configuration of the sensor unit 6. FIG. 5 shows a cross-section of the sensor unit 6. FIG. 5 corresponds to the first example shown in FIG. 2.

The sensor unit 6 includes the optical sensor 60 and the posture sensor 61. The optical sensor 60 and the posture sensor 61 are disposed inside the sensor unit 6 and are fixed to the sensor unit 6.

A hole H1 through which the insertion unit 2 passes is formed in the sensor unit 6. The insertion unit 2 can move in a parallel direction to a center axis CA1 of the insertion unit 2 in the hole H1. In other words, the insertion unit 2 can move in a longitudinal direction D1 of the insertion unit 2 in the hole H1.

In addition, the insertion unit 2 can rotate around the center axis CA1 in the hole H1. For example, when the sensor unit 6 rotates around the center axis CA1, the insertion unit 2 rotates along with the sensor unit 6. When the sensor unit 6 rotates, the insertion unit 2 may be fixed to the sensor unit 6. The sensor unit 6 may include a mechanism that fixes the insertion unit 2 to the sensor unit 6 when the sensor unit 6 rotates. The insertion unit 2 may rotate without the sensor unit 6 rotating. The sensor unit 6 may rotate without the insertion unit 2 rotating. In this case, the endoscope device 1 can determine that the insertion unit 2 has not rotated.

The inner diameter of the hole H1 is almost the same as the outer diameter of the insertion unit 2. For example, the center axis of the hole H1 matches the center axis CA1 of the insertion unit 2. The inner diameter of the hole H1 may be greater than the outer diameter of the insertion unit 2. It is preferable that the inner diameter of the hole H1 be close to the outer diameter of the insertion unit 2 so that each of the optical sensor 60 and the posture sensor 61 can accurately determine a value related to the state of the insertion unit 2.

The optical sensor 60 determines a value related to a moving amount of the insertion unit 2 in the longitudinal direction D1. By doing this, the optical sensor 60 determines a value related to the insertion length of the insertion unit 2. In addition, the optical sensor 60 determines a value related to a rotation amount of the insertion unit 2 around the center axis CA1. The rotation amount corresponds to a relative rotation amount of the insertion unit 2 to the sensor unit 6.

The posture sensor 61 determines a value related to a rotation amount of the sensor unit 6 around the center axis CA1. In a case in which the center axis of the hole H1 matches the center axis CA1 of the insertion unit 2, the posture sensor 61 determines a value related to a rotation amount of the sensor unit 6 around the center axis CA1 by determining the value related to the rotation amount of the sensor unit 6 around the center axis of the hole H1. In addition, the posture sensor 61 determines a value related to the posture of the sensor unit 6. Since the insertion unit 2 is inserted into the hole H1, the value related to the posture of the sensor unit 6 indicates the posture of the insertion unit 2 in the hole H1.

FIG. 6 shows a configuration of the optical sensor 60. The optical sensor 60 includes a light-emitting device 60a and a light-receiving device 60b. The light-emitting device 60a emits light to the insertion unit 2. The light reflected by the surface of the insertion unit 2 is incident on the light-receiving device 60b.

The light-receiving device 60b includes a plurality of light-receiving elements that are two-dimensionally disposed. Many metal wires are woven on the surface of the insertion unit 2. The surface of the insertion unit 2 has a pattern formed by the metal wires. The light-receiving device 60b generates a signal in accordance with the amount of light, thus determining the pattern of the surface of the insertion unit 2 as an image. The optical sensor 60 outputs a signal indicating the image.

The state determination unit 34 calculates a temporal change of the signal output from the optical sensor 60. By doing this, the state determination unit 34 calculates a first moving amount of the insertion unit 2 in the longitudinal direction D1 and calculates a second moving amount of the insertion unit 2 in a perpendicular direction D2 to the longitudinal direction D1. The first moving amount and the second moving amount indicate a relative moving amount of the insertion unit 2 to the sensor unit 6. The first moving amount corresponds to the insertion length of the insertion unit 2. The second moving amount corresponds to the rotation amount of the insertion unit 2 around the center axis CA1.

The posture sensor 61 includes at least one of a 3-axis acceleration sensor, a 3-axis gyro sensor, and a 3-axis geomagnetic sensor similarly to the posture sensor 24. In a case in which the posture sensor 61 includes an acceleration sensor, the posture sensor 61 can determine a physical quantity that is based on the direction of gravity. In a case in which the posture sensor 61 includes a geomagnetic sensor, the posture sensor 61 can determine a physical quantity that is based on the direction of geomagnetism. In a case in which the posture sensor 61 includes a 3-axis acceleration sensor and a 3-axis gyro sensor, the state determination unit 34 can accurately calculate rotation amounts in the directions of three axes, in other words, roll, pitch, and yaw.

The sensor unit 6 may include a locking mechanism to fix the insertion unit 2 to the sensor unit 6. The locking mechanism may be capable of switching between a state in which the insertion unit 2 is fixed to the sensor unit 6 and a state in which the insertion unit 2 can move in the longitudinal direction D1. A user twists the sensor unit 6 with the insertion unit 2 being fixed to the sensor unit 6 and thereby can reduce the amount of force required for twisting the insertion unit 2.

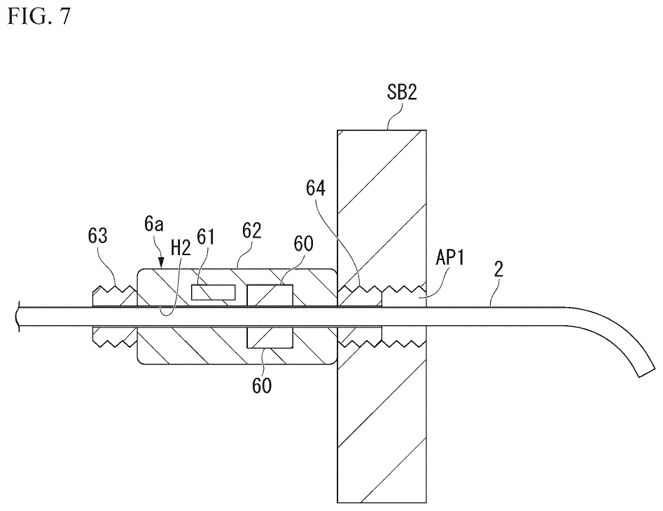

A sensor unit 6a shown in FIG. 7 may be used instead of the sensor unit 6. FIG. 7 shows a configuration of the sensor unit 6a. FIG. 7 shows a cross-section of the sensor unit 6a.

The sensor unit 6a includes a main body unit 62, a screw part 63, and a screw part 64. The main body unit 62 includes the optical sensor 60 and the posture sensor 61. The screw part 63 and the screw part 64 are connected to the main body unit 62. A male screw is formed on the surface of each of the screw parts 63 and 64. A hole H2 through which the insertion unit 2 passes is formed in the main body unit 62, the screw part 63, and the screw part 64.

The insertion unit 2 is inserted into a subject SB2. An access port AP1 is formed in the subject SB2. A female screw is formed in the access port AP1. The male screw of the screw part 64 fits the female screw of the access port AP1, and the sensor unit 6a is fixed to the subject SB2.

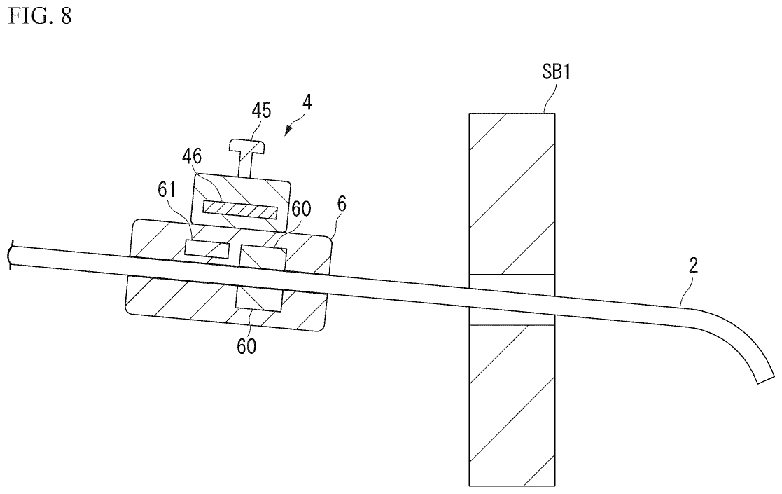

The operation unit 4 may be fixed to the sensor unit 6. FIG. 8 shows a state in which the operation unit 4 is fixed to the sensor unit 6. FIG. 8 shows cross-sections of the operation unit 4 and the sensor unit 6. FIG. 8 corresponds to the second example shown in FIG. 3.

The operation unit 4 includes a joystick 45 and a substrate 46. The joystick 45 accepts a bending operation of bending the bending portion 21. A user inputs a bending instruction to bend the bending portion 21 by operating the joystick 45. The substrate 46 accepts the bending instruction input through the bending operation and outputs the bending instruction to the operation-processing unit 33. The operation-processing unit 33 outputs the bending instruction to the bending control unit 39. The bending control unit 39 controls the motor 38 based on the bending instruction and bends the bending portion 21. The bending control unit 39 determines the bending amount of the bending portion 21.

A user inserts the insertion unit 2 into a subject SB1. The user can perform the bending operation and the insertion operation at the same time. The operation unit 4 may be attachable to and detachable from the sensor unit 6.

A hole through which the insertion unit 2 passes may be formed in the operation unit 4. The operation unit 4 may be fixed to the sensor unit 6, and the insertion unit 2 may pass through the inside of the operation unit 4 and the sensor unit 6.

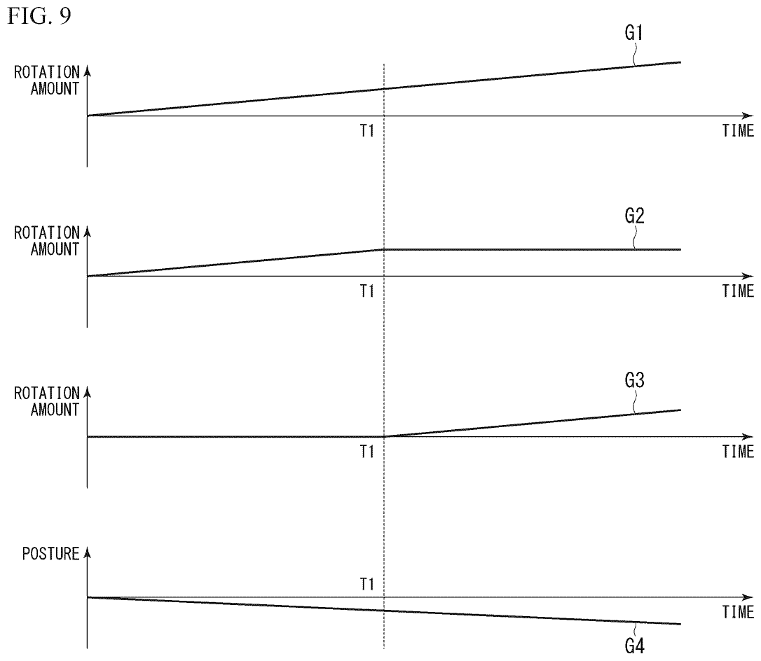

An example of a change of the states of the insertion unit 2 and the sensor unit 6 will be described by using FIG. 9. FIG. 9 shows an example of a change of the states of the insertion unit 2 and the sensor unit 6. FIG. 9 shows graphs of a rotation amount G1, a rotation amount G2, a rotation amount G3, and a posture G4. The horizontal axis of each graph indicates time, and the vertical axis of each graph indicates a rotation amount or a posture.

The rotation amount G1 indicates an absolute rotation amount of the insertion unit 2. The rotation amount G2 indicates a rotation amount of the insertion unit 2 calculated based on the signal output from the optical sensor 60. The rotation amount G2 indicates a relative rotation amount of the insertion unit 2 to the sensor unit 6. The rotation amount G3 indicates a rotation amount of the sensor unit 6 calculated based on the value determined by the posture sensor 61.

The posture G4 indicates a posture of the sensor unit 6 calculated based on the value determined by the posture sensor 61. The posture of the sensor unit 6 is the same as that of the insertion unit 2 in the hole H1 through which the insertion unit 2 passes. For example, the posture G4 indicates the angle of the center axis CA1 of the insertion unit 2 with respect to the horizontal plane. The posture G4 may indicate the angle of the center axis CA1 of the insertion unit 2 with respect to the direction of gravity.

The insertion unit 2 rotates, and the rotation amount G1 gradually increases. The rotation amount G2 increases similarly to the rotation amount G1 before a time point T1. The rotation amount G3 is 0 before the time point T1. In other words, the sensor unit 6 does not rotate. The sensor unit 6 does not rotate, and only the insertion unit 2 rotates before the time point T1.

A user rotates the insertion unit 2 and the sensor unit 6 together in the same direction after the time point T1. Therefore, the rotation amount G2 is constant, and the rotation amount G3 increases. At this time, the rotation amount G3 indicates the rotation amount of the insertion unit 2 and the sensor unit 6.

Since the rotation amount G2 indicates a relative rotation amount of the insertion unit 2 to the sensor unit 6, the rotation amount G2 after the time point T1 is different from the absolute rotation amount G1 of the insertion unit 2. The information-processing unit 40 corrects the rotation amount G2 based on the rotation amount G3, thus calculating the rotation amount G1. Specifically, the information-processing unit 40 adds the rotation amount G3 to the rotation amount G2.

There is a case in which a user adjusts the angle of the insertion unit 2 with respect to a hole (access port) of a subject in order to skilledly insert the insertion unit 2 into the subject. By performing such adjustment, the user can set the direction of the imaging portion 20 inserted into the subject to an intended direction. The posture G4 indicates the posture of the insertion unit 2 outside the subject.

The information-processing unit 40 calculates a corrected rotation amount by using a first rotation amount and a second rotation amount. The first rotation amount indicates a relative rotation amount of the insertion unit 2 to the sensor unit 6. In the example shown in FIG. 9, the first rotation amount corresponds to the rotation amount G2. The second rotation amount indicates the rotation amount of the sensor unit 6. In the example shown in FIG. 9, the second rotation amount corresponds to the rotation amount G3. The corrected rotation amount indicates the absolute rotation amount of the insertion unit 2.

For example, in a case in which the rotation direction (first direction) of the sensor unit 6 determined by the posture sensor 61 is the same as the rotation direction (second direction) of the insertion unit 2 determined by the optical sensor 60, the information-processing unit 40 calculates the corrected rotation amount by adding the second rotation amount to the first rotation amount. In a case in which the first direction is opposite the second direction, the information-processing unit 40 calculates the corrected rotation amount by subtracting the second rotation amount from the first rotation amount.

In a case in which the first direction is opposite the second direction and the second rotation amount is the same as the first rotation amount, the corrected rotation amount is 0. In this case, the insertion unit 2 does not rotate, and only the sensor unit 6 rotates.

In a case in which the second rotation amount is 0, the sensor unit 6 does not rotate. In this case, the information-processing unit 40 acquires the first rotation amount as an accurate rotation amount of the insertion unit 2.

Each of the first and second rotation amounts may have a sign in accordance with the rotation direction. The sign is a positive sign (+) or a negative sign (−). In a case in which the second direction is the same as the first direction, the sign of the second rotation amount is the same as that of the first rotation amount. In a case in which the second direction is opposite the first direction, the sign of the second rotation amount is different from that of the first rotation amount. In a case in which each of the first and second rotation amounts has a sign, the information-processing unit 40 may calculate the corrected rotation amount by adding the second rotation amount to the first rotation amount.

As described above, in a case in which the sensor unit 6 rotates, the information-processing unit 40 corrects the rotation amount of the insertion unit 2 by using the rotation amount of the sensor unit 6. The information-processing unit 40 can calculate an accurate rotation amount of the insertion unit 2.

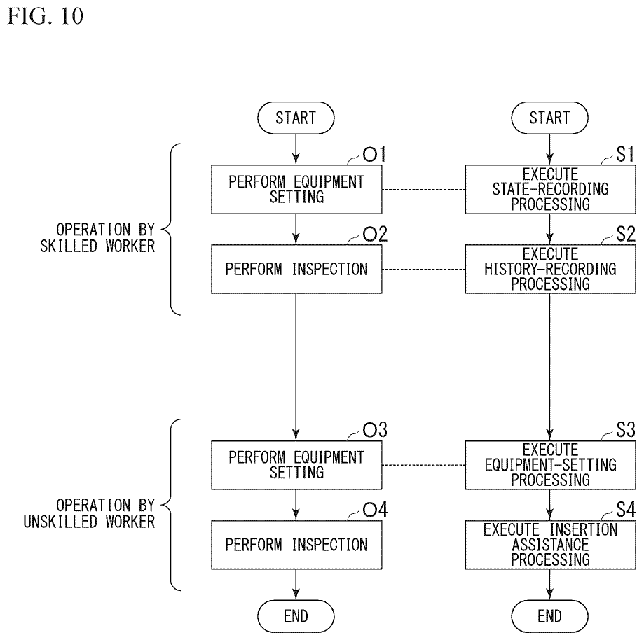

Processing related to the insertion operation will be described by using FIGS. 10 to 19. FIG. 10 shows an entire procedure of the insertion operation.

After a skilled worker who is proficient in inspection performs an inspection, an unskilled worker who is not proficient in inspection performs an inspection. While the skilled worker is performing the inspection, the state of the insertion operation performed by the skilled worker is recorded. The insertion assistance information is generated by using the state. The unskilled worker refers to the insertion assistance information and performs the insertion operation by imitating the insertion operation by the skilled worker. The unskilled worker's imitation of the insertion operation by the skilled worker enhances the efficiency of the inspection.

First, the skilled worker performs equipment setting (operation O1). At this time, the skilled worker sets a positional relationship between the insertion unit 2 and the sensor unit 6 to an initial state and sets a positional relationship between a subject and the insertion unit 2 to an initial state. The endoscope device 1 executes state-recording processing and records the states of the insertion unit 2 and the sensor unit 6 in the initial state (Step S1).

After the equipment setting is performed, the skilled worker performs the insertion operation and performs an inspection (operation O2). The endoscope device 1 executes history-recording processing and records the states of the insertion unit 2 and the sensor unit 6 (Step S2).

After any period has passed since the skilled worker completed work, the unskilled worker performs work. The period may be several days, several weeks, several months, or the like.

First, the unskilled worker performs equipment setting (operation O3). At this time, the unskilled worker sets a positional relationship between the insertion unit 2 and the sensor unit 6 to an initial state and sets a positional relationship between a subject and the insertion unit 2 to an initial state. The endoscope device 1 executes equipment-setting processing. At this time, the endoscope device 1 assists an operation by the unskilled worker to set the state of equipment to an initial state (Step S3).

After the equipment setting is performed, the unskilled worker performs the insertion operation and performs an inspection (operation O4). The endoscope device 1 executes insertion assistance processing and assists the insertion operation by the unskilled worker (Step S4).

For example, the endoscope device 1 has a first mode to learn an operation by the skilled worker and a second mode to assist the insertion operation by the unskilled worker. The endoscope device 1 can switch between the first mode and the second mode by using, for example, a processor.

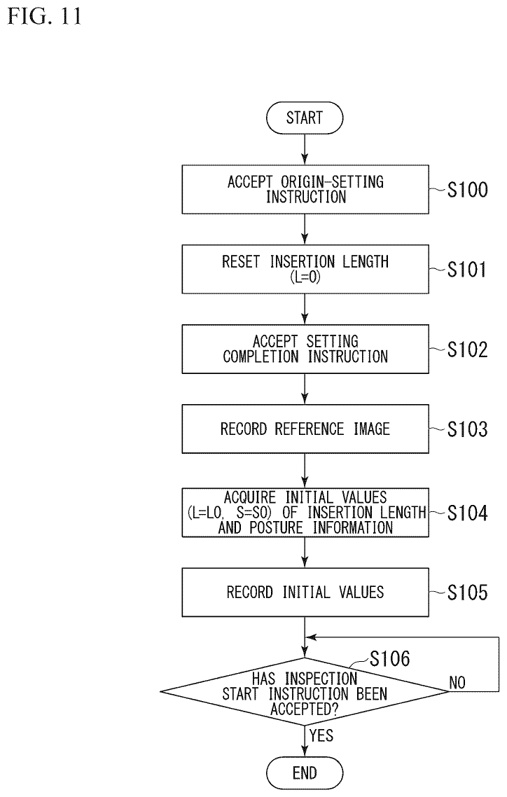

FIG. 11 shows a procedure of the state-recording processing (Step S1) executed by the endoscope device 1 when the skilled worker performs the equipment setting (operation O1). When the first mode is set in the endoscope device 1, the endoscope device 1 executes the state-recording processing.

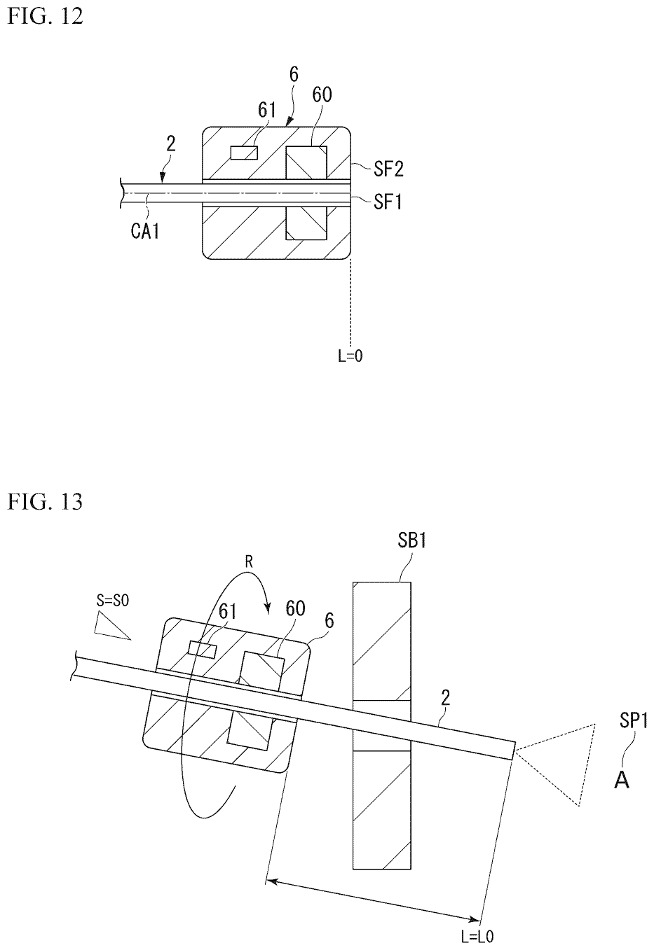

The skilled worker adjusts the position of the insertion unit 2 to the position of the sensor unit 6. FIG. 12 shows a positional relationship between the insertion unit 2 and the sensor unit 6 at this time. FIG. 12 shows a cross-section of the sensor unit 6. For example, the skilled worker matches a distal end surface SF1 of the insertion unit 2 to an end surface SF2 of the sensor unit 6. At this time, the skilled worker inputs an origin-setting instruction into the endoscope device 1 by operating the operation unit 4.

The operation-processing unit 33 outputs the origin-setting instruction to the state determination unit 34. The state determination unit 34 accepts the origin-setting instruction (Step S100).

After Step S100, the state determination unit 34 resets the insertion length calculated based on the value output from the optical sensor 60 to 0 (Step S101). After the insertion length is reset to 0, a newly calculated insertion length indicates a moving amount of the insertion unit 2 in the longitudinal direction D1 of the insertion unit 2 after Step S101.

The skilled worker inserts the insertion unit 2 into a subject and sets a relative state of the insertion unit 2 to the subject to an initial state. FIG. 13 shows a positional relationship between a subject SB1 and the insertion unit 2 at this time. FIG. 13 shows cross-sections of the subject SB1 and the sensor unit 6.

The skilled worker adjusts the position of the insertion unit 2 so that the imaging device 23 can acquire an image of a portion SP1 in the subject SB1. In addition, the skilled worker adjusts the posture of the insertion unit 2 so that the insertion unit 2 can smoothly advance.

While the skilled worker is performing the above-described adjustment, the state determination unit 34 acquires a value determined by each of the optical sensor 60 and the posture sensor 61. The state determination unit 34 calculates an insertion length of the insertion unit 2. In addition, the state determination unit 34 calculates a posture of the sensor unit 6 and generates posture information of the sensor unit 6.

After the state of the insertion unit 2 is set to an intended state, the skilled worker inputs a setting completion instruction into the endoscope device 1 by operating the operation unit 4. The operation-processing unit 33 outputs the setting completion instruction to the information-processing unit 40. The information-processing unit 40 accepts the setting completion instruction (Step S102).

After Step S102, the information-processing unit 40 acquires an image processed by the image-processing unit 30 and records the image as a reference image on the memory 41 (Step S103).

After Step S103, the information-processing unit 40 acquires the insertion length of the insertion unit 2 and the posture information of the sensor unit 6 from the state determination unit 34 (Step S104). For example, when the state of the insertion unit 2 is set to the state shown in FIG. 13, the insertion length of the insertion unit 2 is L0 and the value indicating the posture (slope) of the sensor unit 6 is S0.

After Step S104, the information-processing unit 40 records the insertion length of the insertion unit 2 and the posture information of the sensor unit 6 on the memory 41 (Step S105).

The skilled worker inputs an inspection start instruction into the endoscope device 1 by operating the operation unit 4 in order to start an inspection. The operation-processing unit 33 outputs the inspection start instruction to the state determination unit 34, the posture determination unit 35, and the information-processing unit 40. The state determination unit 34, the posture determination unit 35, and the information-processing unit 40 accept the inspection start instruction (Step S106). When the inspection start instruction has been accepted, the state-recording processing shown in FIG. 11 is completed.

The insertion length is calculated based on the value output from the optical sensor 60. In a case in which the distance between the sensor unit 6 and a subject changes, a change of the distance may be erroneously determined as a moving amount of the insertion unit 2 and the insertion length may contain an error. It is preferable that the distance between the sensor unit 6 and the subject be kept constant after the insertion length is reset in Step S101. Therefore, the sensor unit 6 may include a distance sensor that measures the distance between the sensor unit 6 and the subject. The state determination unit 34 may output distance information indicating the distance to the display unit 5 via the image-processing unit 30. The skilled worker may refer to the distance information displayed on the display unit 5 and may keep the distance between the sensor unit 6 and the subject constant.

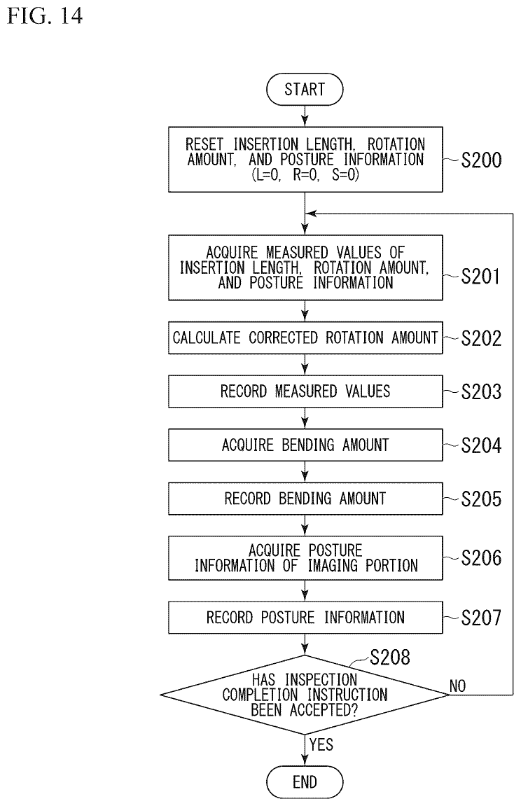

FIG. 14 shows a procedure of the history-recording processing (Step S2) executed by the endoscope device 1 when the skilled worker performs the inspection (operation O2).

The state determination unit 34, the posture determination unit 35, and the information-processing unit 40 reset various values (Step S200). The state determination unit 34, the posture determination unit 35, and the information-processing unit 40 execute the following processing in Step S200.

The state determination unit 34 resets the insertion length calculated based on the value output from the optical sensor 60 to 0. The state determination unit 34 resets the posture calculated based on the value output from the posture sensor 61 to 0. The posture determination unit 35 resets the posture calculated based on the value output from the posture sensor 24 to 0.

After the insertion length is reset to 0, a newly calculated insertion length indicates a moving amount of the insertion unit 2 in the longitudinal direction D1 of the insertion unit 2 after Step S200. After the posture of the sensor unit 6 is reset to 0, a newly calculated posture indicates an amount of a change of the posture of the sensor unit 6 after Step S200. After the posture of the imaging portion 20 is reset to 0, a newly calculated posture indicates an amount of a change of the posture of the imaging portion 20 after Step S200.

The information-processing unit 40 acquires the rotation amount of the insertion unit 2 and the rotation amount of the sensor unit 6 from the state determination unit 34. The information-processing unit 40 calculates a corrected rotation amount by using the rotation amount of the insertion unit 2 and the rotation amount of the sensor unit 6. The corrected rotation amount indicates an absolute rotation amount of the insertion unit 2. The information-processing unit 40 converts the corrected rotation amount that has been calculated into 0, thus resetting the corrected rotation amount to 0. The information-processing unit 40 holds a conversion expression used in this conversion.

The skilled worker performs the insertion operation and causes the insertion unit 2 to advance in a subject. The state determination unit 34 acquires the value determined by each of the optical sensor 60 and the posture sensor 61. The state determination unit 34 calculates an insertion length of the insertion unit 2, a rotation amount of the insertion unit 2, and a rotation amount of the sensor unit 6. The state determination unit 34 calculates a posture of the sensor unit 6 and generates posture information of the sensor unit 6. The posture determination unit 35 acquires the value determined by the posture sensor 24. The posture determination unit 35 calculates a posture of the imaging portion 20 and generates posture information of the imaging portion 20.

After Step S200, the information-processing unit 40 acquires the insertion length of the insertion unit 2, the rotation amount of the insertion unit 2, the rotation amount of the sensor unit 6, and the posture information of the sensor unit 6 from the state determination unit 34 (Step S201).

After Step S201, the information-processing unit 40 calculates a corrected rotation amount by using the rotation amount of the insertion unit 2 and the rotation amount of the sensor unit 6. The information-processing unit 40 converts the corrected rotation amount into a new value by using the conversion expression used in Step S200 (Step S202). The new value indicates a change of the corrected rotation amount after Step S200 and is used as a corrected rotation amount in processing after Step S202.

After Step S202, the information-processing unit 40 records the insertion length of the insertion unit 2, the corrected rotation amount, and the posture information of the sensor unit 6 on the memory 41 as insertion state information (Step S203). The insertion length of the insertion unit 2, the corrected rotation amount, and the posture information of the sensor unit 6 are associated with time information.

After Step S203, the information-processing unit 40 acquires the bending amount of the bending portion 21 from the bending control unit 39 (Step S204). For example, the bending amount indicates a bending amount in the upward (U) or downward (D) direction and indicates a bending amount in the left (L) or right (R) direction.

After Step S204, the information-processing unit 40 records the bending amount of the bending portion 21 on the memory 41 (Step S205). The bending amount is included in the insertion state information and is associated with the time information.

After Step S205, the information-processing unit 40 acquires the posture information of the imaging portion 20 from the posture determination unit 35 (Step S206).

After Step S206, the information-processing unit 40 records the posture information of the imaging portion 20 on the memory 41 (Step S207). The posture information is included in the insertion state information and is associated with the time information.

When the inspection is completed, the skilled worker inputs an inspection completion instruction into the endoscope device 1 by operating the operation unit 4. The operation-processing unit 33 outputs the inspection completion instruction to the state determination unit 34, the posture determination unit 35, and the information-processing unit 40. The state determination unit 34, the posture determination unit 35, and the information-processing unit 40 accept the inspection completion instruction (Step S208).

When the inspection completion instruction has been accepted, the history-recording processing shown in FIG. 14 is completed. Steps S201 to S207 are repeated until the inspection completion instruction is accepted.

Step S202 and Step S203 may be executed at any timing between Step S201 and Step S208. Step S204 and Step S205 may be executed at any timing between Step S200 and Step S208. Step S206 and Step S207 may be executed at any timing between Step S200 and Step S208.

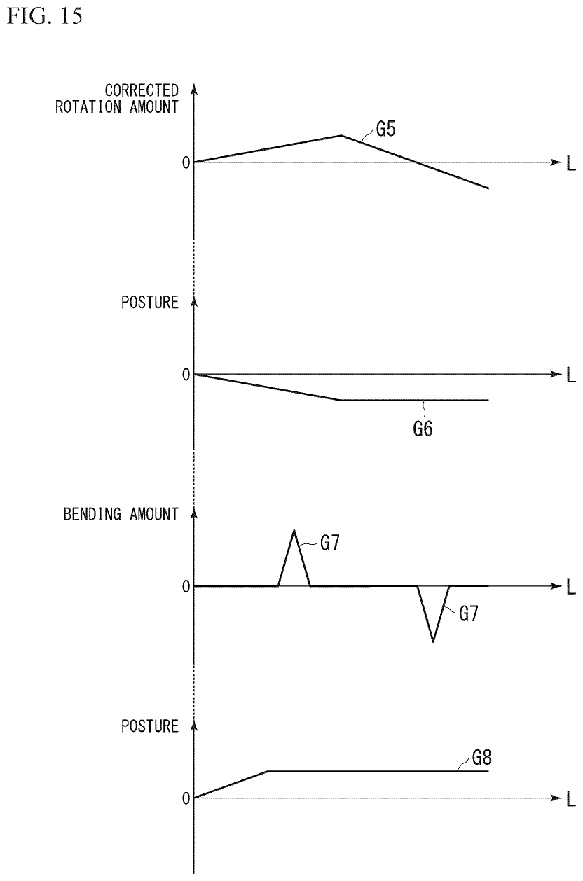

An example of a change of the states of the insertion unit 2 and the sensor unit 6 will be described by using FIG. 15. FIG. 15 shows an example of a change of the states of the insertion unit 2 and the sensor unit 6. FIG. 15 shows graphs of a corrected rotation amount G5, a posture G6, a bending amount G7, and a posture G8. The horizontal axis of each graph indicates an insertion length, and the vertical axis of each graph indicates a rotation amount or the like.

The corrected rotation amount G5 indicates a corrected rotation amount calculated by using the rotation amount of the insertion unit 2 and the rotation amount of the sensor unit 6. The posture G6 indicates a posture of the sensor unit 6 calculated based on the value determined by the posture sensor 61. The posture of the sensor unit 6 is the same as that of the insertion unit 2 in the hole H1 through which the insertion unit 2 passes. For example, the posture G6 indicates the angle of the center axis CA1 of the insertion unit 2 with respect to the horizontal plane. The posture G6 may indicate the angle of the center axis CA1 of the insertion unit 2 with respect to the direction of gravity. For example, the bending amount G7 indicates a bending amount of the bending portion 21 in each of the upward (U) and downward (D) directions. The posture G8 indicates a posture of the imaging portion 20 calculated based on the value determined by the posture sensor 24.

The insertion length, the corrected rotation amount G5, the posture G6, the bending amount G7, and the posture G8 are associated with each other by the same time information. Therefore, the information-processing unit 40 can convert the corrected rotation amount G5, the posture G6, the bending amount G7, and the posture G8 into values in accordance with the insertion length. The insertion state information recorded on the memory 41 includes the rotation amount of the insertion unit 2, the rotation amount of the sensor unit 6, the corrected rotation amount, the posture information of the sensor unit 6, the bending amount of the bending portion 21, and the posture information of the imaging portion 20. These are associated with the insertion length.

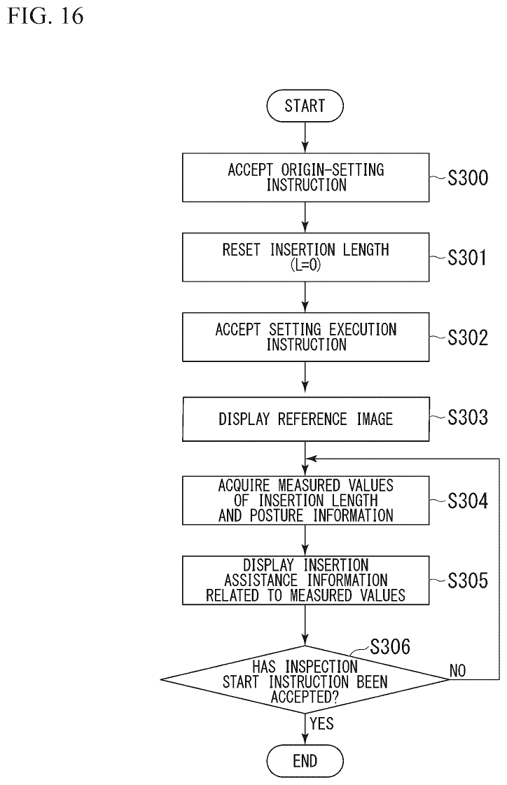

FIG. 16 shows a procedure of the equipment-setting processing (Step S3) executed by the endoscope device 1 when the unskilled worker performs the equipment setting (operation O3). When the second mode is set in the endoscope device 1, the endoscope device 1 executes the equipment-setting processing.

The unskilled worker adjusts the position of the insertion unit 2 to the position of the sensor unit 6. At this time, the work performed by the unskilled worker is similar to that (FIG. 12) performed by the skilled worker. For example, the unskilled worker matches the distal end surface of the insertion unit 2 to the end surface of the sensor unit 6. At this time, the unskilled worker inputs an origin-setting instruction into the endoscope device 1 by operating the operation unit 4.

The operation-processing unit 33 outputs the origin-setting instruction to the state determination unit 34. The state determination unit 34 accepts the origin-setting instruction (Step S300).

After Step S300, the state determination unit 34 resets the insertion length calculated based on the value output from the optical sensor 60 to 0 (Step S301). After the insertion length is reset to 0, a newly calculated insertion length indicates a moving amount of the insertion unit 2 in the longitudinal direction D1 of the insertion unit 2 after Step S301.

The unskilled worker inserts the insertion unit 2 into a subject and sets a relative state of the insertion unit 2 to the subject to an initial state. At this time, the unskilled worker performs work such that the same state as the initial state set by the skilled worker is realized. The endoscope device 1 executes processing of assisting the work performed by the unskilled worker. Hereinafter, details of the processing will be described.

The unskilled worker inputs a setting execution instruction into the endoscope device 1 by operating the operation unit 4. The operation-processing unit 33 outputs the setting execution instruction to the insertion assistance unit 42. The insertion assistance unit 42 accepts the setting execution instruction (Step S302).

The insertion assistance unit 42 acquires the reference image recorded on the memory 41 in Step S103 and outputs the reference image to the display unit 5 via the image-processing unit 30. The display unit 5 displays the reference image (Step S303).

The state determination unit 34 acquires the value determined by each of the optical sensor 60 and the posture sensor 61. The state determination unit 34 calculates an insertion length of the insertion unit 2. In addition, the state determination unit 34 calculates a posture of the sensor unit 6 and generates posture information of the sensor unit 6.

After Step S303, the insertion assistance unit 42 acquires the insertion length of the insertion unit 2 and the posture information of the sensor unit 6 from the state determination unit 34 (Step S304).

After Step S304, the insertion assistance unit 42 acquires the information recorded on the memory 41 in Step S105. In other words, the insertion assistance unit 42 acquires the insertion length (L0) of the insertion unit 2 and the posture information (S0) of the sensor unit 6. The insertion assistance unit 42 generates insertion assistance information by using the information acquired from the memory 41 and the information acquired from the state determination unit 34 in Step S304. The insertion assistance unit 42 outputs the insertion assistance information to the display unit 5 via the image-processing unit 30. The display unit 5 displays the insertion assistance information related to the insertion length and the posture information (Step S305).

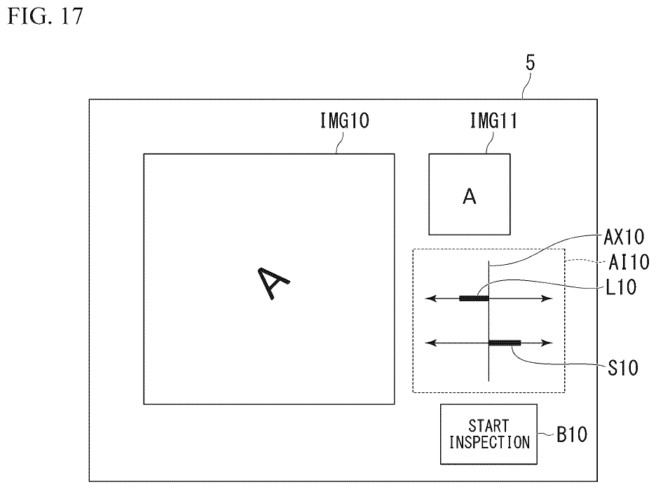

FIG. 17 shows information displayed on the display unit 5. The display unit 5 displays a live image IMG10, a reference image IMG11, insertion assistance information AI10, and a button B10.

The live image IMG10 is a present image generated in real time by the imaging device 23. The reference image IMG11 is acquired from the memory 41 in Step S303.

The insertion assistance information AI10 includes insertion length information L10. The insertion length information L10 indicates the difference between the previous insertion length (L0) and the present insertion length. The previous insertion length (L0) is acquired from the memory 41 in Step S305. The present insertion length is acquired from the state determination unit 34 in Step S304. The insertion length information L10 is displayed as a line having the length in accordance with the amount of the difference. The insertion length information L10 is displayed on the right or left side of an axis AX10 in accordance with a relationship of the amount between the previous insertion length (L0) and the present insertion length.

The insertion assistance information AI10 includes posture information S10. The posture information S10 indicates the difference between the previous value (S0) of the posture information of the sensor unit 6 and the present value of the posture information of the sensor unit 6. The previous value (S0) of the posture information is acquired from the memory 41 in Step S305. The present value of the posture information is acquired from the state determination unit 34 in Step S304. The posture information S10 is displayed as a line having the length in accordance with the amount of the difference. The posture information S10 is displayed on the right or left side of the axis AX10 in accordance with a relationship of the amount between the previous value (S0) of the posture information and the present value of the posture information. The posture information S10 indicates the posture of the insertion unit 2 in the hole H1 through which the insertion unit 2 passes.

The unskilled worker compares the live image IMG10 with the reference image IMG11. The unskilled worker adjusts the rotation amount of the insertion unit 2 such that the composition of the live image IMG10 matches the composition of the reference image IMG11. In addition, the unskilled worker refers to the insertion assistance information AI10. The unskilled worker adjusts the position of the insertion unit 2 such that the difference corresponding to the insertion length information L10 matches 0. The unskilled worker adjusts the posture of the insertion unit 2 such that the difference corresponding to the posture information S10 matches 0. The insertion length information L10 and the posture information S10 are updated in accordance with the operation performed by the unskilled worker while the unskilled worker is adjusting the rotation amount, the position, and the posture of the insertion unit 2.

When the above-described adjustment is completed, the unskilled worker inputs an inspection start instruction into the endoscope device 1 by operating the operation unit 4 in order to start an inspection. For example, the unskilled worker presses the button B10 by operating the operation unit 4. By doing this, the unskilled worker can input the inspection start instruction into the endoscope device 1.

The operation-processing unit 33 outputs the inspection start instruction to the state determination unit 34, the posture determination unit 35, and the insertion assistance unit 42. The state determination unit 34, the posture determination unit 35, and the insertion assistance unit 42 accept the inspection start instruction (Step S306). When the inspection start instruction has been accepted, the equipment-setting processing shown in FIG. 16 is completed. Step S304 and Step S305 are repeated until the inspection start instruction is accepted.

The insertion assistance unit 42 may determine whether the present insertion length matches the previous insertion length (L0) and the present value of the posture information matches the previous value (S0) of the posture information. When the insertion assistance unit 42 determines that the present insertion length matches the previous insertion length (L0) and the present value of the posture information matches the previous value (S0) of the posture information, the insertion assistance unit 42 may automatically accept the inspection start instruction and may output the inspection start instruction to the state determination unit 34 and the posture determination unit 35.

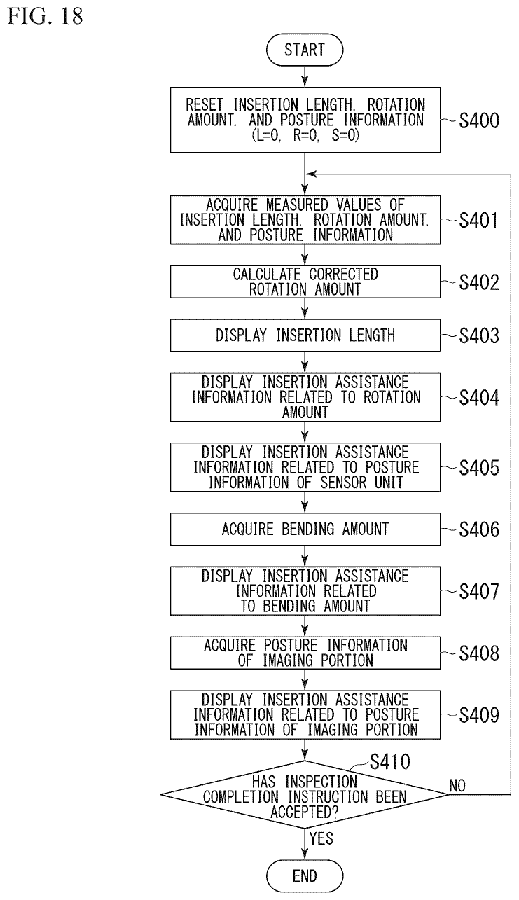

FIG. 18 shows a procedure of the insertion assistance processing (Step S4) executed by the endoscope device 1 when the unskilled worker performs the inspection (operation O4).

The state determination unit 34, the posture determination unit 35, and the information-processing unit 40 reset various values (Step S400). The state determination unit 34, the posture determination unit 35, and the information-processing unit 40 execute the following processing in Step S400.

The state determination unit 34 resets the insertion length calculated based on the value output from the optical sensor 60 to 0. The state determination unit 34 resets the posture calculated based on the value output from the posture sensor 61 to 0. The posture determination unit 35 resets the posture calculated based on the value output from the posture sensor 24 to 0.

After the insertion length is reset to 0, a newly calculated insertion length indicates a moving amount of the insertion unit 2 in the longitudinal direction D1 of the insertion unit 2 after Step S400. After the posture of the sensor unit 6 is reset to 0, a newly calculated posture indicates an amount of a change of the posture of the sensor unit 6 after Step S400. After the posture of the imaging portion 20 is reset to 0, a newly calculated posture indicates an amount of a change of the posture of the imaging portion 20 after Step S400.

The information-processing unit 40 acquires the rotation amount of the insertion unit 2 and the rotation amount of the sensor unit 6 from the state determination unit 34. The information-processing unit 40 calculates a corrected rotation amount by using the rotation amount of the insertion unit 2 and the rotation amount of the sensor unit 6. The corrected rotation amount indicates an absolute rotation amount of the insertion unit 2. The information-processing unit 40 converts the corrected rotation amount that has been calculated into 0, thus resetting the corrected rotation amount to 0. The information-processing unit 40 holds a conversion expression used in this conversion.

The unskilled worker performs the insertion operation and causes the insertion unit 2 to advance in a subject. The state determination unit 34 acquires the value determined by each of the optical sensor 60 and the posture sensor 61. The state determination unit 34 calculates an insertion length of the insertion unit 2, a rotation amount of the insertion unit 2, and a rotation amount of the sensor unit 6. The state determination unit 34 calculates a posture of the sensor unit 6 and generates posture information of the sensor unit 6. The posture determination unit 35 acquires the value determined by the posture sensor 24. The posture determination unit 35 calculates a posture of the imaging portion 20 and generates posture information of the imaging portion 20.

After Step S400, the insertion assistance unit 42 acquires the insertion length of the insertion unit 2, the rotation amount of the insertion unit 2, the rotation amount of the sensor unit 6, and the posture information of the sensor unit 6 from the state determination unit 34 (Step S401).

After Step S401, the information-processing unit 40 acquires the rotation amount of the insertion unit 2 and the rotation amount of the sensor unit 6 from the state determination unit 34. The information-processing unit 40 calculates a corrected rotation amount by using the rotation amount of the insertion unit 2 and the rotation amount of the sensor unit 6. The information-processing unit 40 converts the corrected rotation amount into a new value by using the conversion expression used in Step S400 (Step S402). The new value indicates a change of the corrected rotation amount after Step S400 and is used as a corrected rotation amount in processing after Step S402.

After Step S402, the insertion assistance unit 42 outputs the insertion length acquired from the state determination unit 34 in Step S401 to the display unit 5 via the image-processing unit 30. The display unit 5 displays the insertion length (Step S403).

After Step S403, the insertion assistance unit 42 acquires the corrected rotation amount recorded on the memory 41 in Step S203. At this time, the insertion assistance unit 42 acquires the corrected rotation amount associated with the same insertion length as that acquired from the state determination unit 34 in Step S401. The insertion assistance unit 42 acquires the corrected rotation amount calculated in Step S402 from the information-processing unit 40. The insertion assistance unit 42 generates insertion assistance information by using the corrected rotation amount acquired from the memory 41 and the corrected rotation amount calculated in real time in Step S402. For example, the insertion assistance unit 42 calculates the difference between the two corrected rotation amounts. The insertion assistance unit 42 generates insertion assistance information in accordance with the difference. The insertion assistance unit 42 outputs the insertion assistance information to the display unit 5 via the image-processing unit 30. The display unit 5 displays the insertion assistance information related to the rotation amount of the insertion unit 2 (Step S404).

After Step S404, the insertion assistance unit 42 acquires the posture information of the sensor unit 6 recorded on the memory 41 in Step S203. At this time, the insertion assistance unit 42 acquires the posture information associated with the same insertion length as that acquired from the state determination unit 34 in Step S401. The insertion assistance unit 42 generates insertion assistance information by using the posture information acquired from the memory 41 and the posture information acquired from the state determination unit 34 in Step S401. The insertion assistance unit 42 outputs the insertion assistance information to the display unit 5 via the image-processing unit 30. The display unit 5 displays the insertion assistance information related to the posture information of the sensor unit 6 (Step S405).

After Step S405, the insertion assistance unit 42 acquires the bending amount of the bending portion 21 from the bending control unit 39 (Step S406). For example, the bending amount indicates a bending amount in the upward (U) or downward (D) direction and indicates a bending amount in the left (L) or right (R) direction.

After Step S406, the insertion assistance unit 42 acquires the bending amount recorded on the memory 41 in Step S205. At this time, the insertion assistance unit 42 acquires the bending amount associated with the same insertion length as that acquired from the state determination unit 34 in Step S401. The insertion assistance unit 42 generates insertion assistance information by using the bending amount acquired from the memory 41 and the bending amount acquired from the bending control unit 39 in Step S406. The insertion assistance unit 42 outputs the insertion assistance information to the display unit 5 via the image-processing unit 30. The display unit 5 displays the insertion assistance information related to the bending amount (Step S407).

After Step S407, the insertion assistance unit 42 acquires the posture information of the imaging portion 20 from the posture determination unit 35 (Step S408).

After Step S408, the insertion assistance unit 42 acquires the posture information of the imaging portion 20 recorded on the memory 41 in Step S207. At this time, the insertion assistance unit 42 acquires the posture information associated with the same insertion length as that acquired from the state determination unit 34 in Step S401. The insertion assistance unit 42 generates insertion assistance information by using the posture information acquired from the memory 41 and the posture information acquired from the posture determination unit 35 in Step S408. The insertion assistance unit 42 outputs the insertion assistance information to the display unit 5 via the image-processing unit 30. The display unit 5 displays the insertion assistance information related to the posture information of the imaging portion 20 (Step S409).

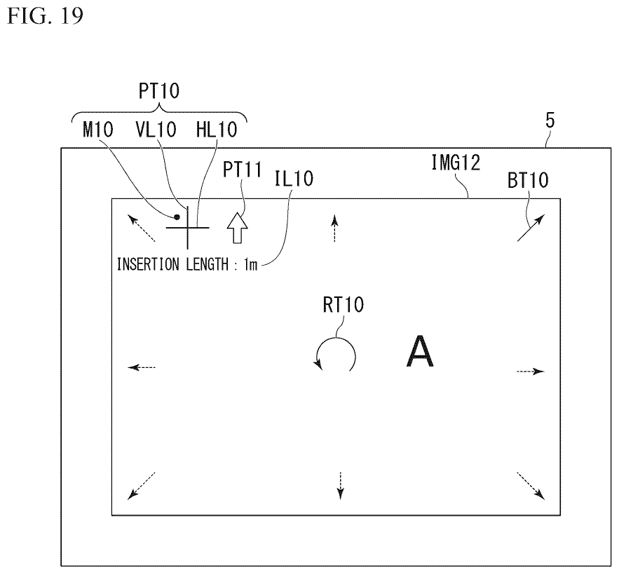

FIG. 19 shows information displayed on the display unit 5. The display unit 5 displays a live image IMG12, an insertion length IL10, a rotation target RT10, a posture target PT10, a bending target BT10, and a posture target PT11.

The live image IMG12 is a present image generated in real time by the imaging device 23. The insertion length IL10, the rotation target RT10, the posture target PT10, the bending target BT10, and the posture target PT11 are displayed on the live image IMG12.

The insertion length IL10 Is displayed in step S403.

The rotation target RT10 indicates a target of the rotation amount of the insertion unit 2. The rotation target RT10 corresponds to the insertion assistance information displayed in Step S404. For example, the rotation target RT10 is displayed as an arrow in accordance with the difference calculated in Step S404. The direction of the arrow corresponds to a positive or negative sign of the difference. The length of the arrow corresponds to the amount of the difference. The arrow may be displayed in a color corresponding to the amount of the difference. The arrow may have the thickness corresponding to the amount of the difference.

A method of displaying a target of the rotation amount of the insertion unit 2 is not limited to that shown in FIG. 19. For example, the insertion assistance unit 42 may display the corrected rotation amount recorded on the memory 41 in Step S203 and the corrected rotation amount calculated in Step S402 on the display unit 5.

The posture target PT10 indicates a target of the posture of the sensor unit 6. The posture of the sensor unit 6 is the same as that of the insertion unit 2 in the hole H1 through which the insertion unit 2 passes. The posture target PT10 corresponds to the insertion assistance information displayed in Step S405. The posture target PT10 includes a line VL10, a line HL10, and a mark M10.

The line VL10 indicates the value of the posture information of the sensor unit 6 in the vertical direction. The line HL10 indicates the value of the posture information of the sensor unit 6 in the horizontal direction. The intersection of the line VL10 and the line HL10 indicates the value of the posture information recorded on the memory 41 in Step S203. The mark M10 indicates the present value of the posture information of the sensor unit 6. The vertical position of the mark M10 is in accordance with the difference between the present value of the posture information in the vertical direction and the previous value of the posture information in the vertical direction. The horizontal position of the mark M10 is in accordance with the difference between the present value of the posture information in the horizontal direction and the previous value of the posture information in the horizontal direction.

A method of displaying a target of the posture of the sensor unit 6 is not limited to that shown in FIG. 19. For example, the insertion assistance unit 42 may calculate the difference between the value of the posture information recorded on the memory 41 in Step S203 and the present value of the posture information. The insertion assistance unit 42 may display an arrow having the length in accordance with the amount of the difference on the display unit 5.

The bending target BT10 indicates a target of the bending amount of the bending portion 21. The insertion assistance unit 42 refers to the bending amount (first bending amount) recorded on the memory 41 in Step S205 and refers to the bending amount (second bending amount) acquired from the bending control unit 39 in Step S406. The insertion assistance unit 42 calculates a bending direction and a bending amount required for changing the state of the bending portion 21 from a state having the second bending amount to a state having the first bending amount.

The insertion assistance unit 42 displays an arrow indicating the calculated bending direction and bending amount as the bending target BT10 on the display unit 5. The direction of the arrow indicates the bending direction. The length of the arrow indicates the bending amount. The arrow may be displayed in a color corresponding to the bending amount. The arrow may have the thickness corresponding to the bending amount.

A method of displaying a target of the bending amount of the bending portion 21 is not limited to that shown in FIG. 19. For example, the insertion assistance unit 42 may display an arrow indicating the first bending amount and an arrow indicating the second bending amount on the display unit 5.

The posture target PT11 indicates a target of the posture of the imaging portion 20. The posture target PT11 corresponds to the insertion assistance information displayed in Step S409. For example, the insertion assistance unit 42 refers to the posture information (first posture information) recorded on the memory 41 in Step S207 and the posture information (second posture information) acquired from the posture determination unit 35 in Step S408. The insertion assistance unit 42 calculates the difference (first difference) between a vertical component of the first posture information and a vertical component of the second posture information. In addition, the insertion assistance unit 42 calculates the difference (second difference) between a horizontal component of the first posture information and a horizontal component of the second posture information.

The insertion assistance unit 42 displays a first arrow having the length corresponding to the first difference on the display unit 5 and displays a second arrow having the length corresponding to the second difference on the display unit 5. In the example shown in FIG. 19, the second difference is 0. Therefore, the second arrow is not displayed, and only the first arrow is displayed. The first arrow may be displayed in a color corresponding to the amount of the first difference or may have the thickness corresponding to the amount of the first difference. The second arrow may be displayed in a color corresponding to the amount of the second difference or may have the thickness corresponding to the amount of the second difference.

A method of displaying a target of the posture of the imaging portion 20 is not limited to that shown in FIG. 19. For example, the insertion assistance unit 42 may use a similar method to that of displaying the posture target PT10.

The unskilled worker refers to the information shown in FIG. 19 and adjusts the rotation amount of the insertion unit 2 and the like. The unskilled worker adjusts the rotation amount of the insertion unit 2 in accordance with the rotation target RT10. The unskilled worker adjusts the posture of the sensor unit 6 in accordance with the posture target PT10. The unskilled worker adjusts the bending amount of the bending portion 21 in accordance with the bending target BT10.

After the rotation amount of the insertion unit 2, the posture of the sensor unit 6, and the bending amount of the bending portion 21 are adjusted, the unskilled worker checks the direction of the insertion unit 2 in accordance with the posture target PT11. There is a case in which a path through which the insertion unit 2 passes branches into two or more paths. When the present posture of the imaging portion 20 is different from a target posture of the imaging portion 20, the insertion unit 2 may be inserted into an erroneous path. In such a case, the unskilled worker can put the insertion unit 2 back to a branch portion and can insert the insertion unit 2 into a correct path.

When the inspection is completed, the unskilled worker inputs an inspection completion instruction into the endoscope device 1 by operating the operation unit 4. The operation-processing unit 33 outputs the inspection completion instruction to the state determination unit 34, the posture determination unit 35, and the insertion assistance unit 42. The state determination unit 34, the posture determination unit 35, and the insertion assistance unit 42 accept the inspection completion instruction (Step S410).

When the inspection completion instruction has been accepted, the insertion assistance processing shown in FIG. 18 is completed. Steps S401 to S409 are repeated until the inspection completion instruction is accepted.

Step S403 may be executed at any timing between Step S401 and Step S410. Step S402 and Step S404 may be executed at any timing between Step S401 and Step S410. Step S405 may be executed at any timing between Step S401 and Step S410. Step S406 and Step S407 may be executed at any timing between Step S400 and Step S410. Step S408 and Step S409 may be executed at any timing between Step S400 and Step S410.

The insertion assistance unit 42 may display a three-dimensional model of the insertion unit 2 on the display unit 5. The insertion assistance unit 42 may display a target of the rotation amount of the insertion unit 2 and a target of the posture of the insertion unit 2 on the three-dimensional model. Due to this, visibility of information required for adjusting the rotation amount and the posture of the insertion unit 2 is improved.

The bending control unit 39 may bend the bending portion 21 regardless of the bending operation performed by a user. For example, the insertion assistance unit 42 calculates a bending direction and a bending amount required for changing the state of the bending portion 21 from a state having the second bending amount to a state having the first bending amount. The insertion assistance unit 42 outputs a bending instruction including the bending direction and the bending amount to the bending control unit 39. The bending control unit 39 bends the bending portion 21 based on the bending instruction.

There is a case in which the rotation state of the insertion unit 2 in an inspection performed by the unskilled worker is different from that of the insertion unit 2 in a previous inspection performed by the skilled worker. In such a case, it is difficult for the unskilled worker to perform a correct bending operation. Therefore, the insertion assistance unit 42 may calculate a bending direction and a bending amount required for bringing the rotation direction of the insertion unit 2 closer to a rotation direction in the previous inspection and bringing the rotation amount of the insertion unit 2 closer to a rotation amount in the previous inspection. The insertion assistance unit 42 may output a bending instruction including the bending direction and the bending amount to the bending control unit 39. The bending control unit 39 may bend the bending portion 21 based on the bending instruction. Since the unskilled worker does not need to care for both the rotation amount of the insertion unit 2 and the bending amount of the bending portion 21, the operability is improved.

The state determination unit 34 may output the insertion length of the insertion unit 2, the rotation amount of the insertion unit 2, the rotation amount of the sensor unit 6, and the posture information of the sensor unit 6 to the external IF 32. The posture determination unit 35 may output the posture information of the imaging portion 20 to the external IF 32. The external IF 32 may transmit these pieces of information to the external PC 8.

The external PC 8 may execute Step S105, Step S202, Step S203, Step S205, and Step S207. The external PC 8 may generate insertion assistance information in Step S305 and may transmit the generated insertion assistance information to the endoscope device 1. The external PC 8 may calculate a corrected rotation amount in Step S402. The external PC 8 may generate insertion assistance information in Step S404, Step S405, Step S407, and Step S409 and may transmit the generated insertion assistance information to the endoscope device 1. A server or the like may be used instead of the external PC 8.

An inspection state determination system (endoscope device 1) according to each aspect of the present invention includes the sensor unit 6, the posture sensor 61 (second sensor), the information-processing unit 40 (control unit), and the insertion assistance unit 42 (control unit). The sensor unit 6 includes the optical sensor 60 (first sensor) that determines a first rotation amount indicating the rotation amount of the elongated insertion unit 2 of the endoscope device 1 around the center axis CA1 of the insertion unit 2 when the insertion unit 2 is inserted into a subject. The hole H1 through which the insertion unit 2 passes is formed in the sensor unit 6. The posture sensor 61 is disposed in the sensor unit 6. The posture sensor 61 determines a second rotation amount indicating the rotation amount of the sensor unit 6 around the center axis CA1 when the insertion unit 2 is inserted into the subject. The information-processing unit 40 acquires the first rotation amount and the second rotation amount. The information-processing unit 40 calculates a corrected rotation amount by correcting the first rotation amount based on the second rotation amount.

An inspection state determination method according to each aspect of the present invention includes a first acquisition step, a second acquisition step, and a calculation step. The information-processing unit 40 (control unit) acquires the first rotation amount in the first acquisition step (Step S401). The information-processing unit 40 acquires the second rotation amount in the second acquisition step (Step S401). The information-processing unit 40 calculates a corrected rotation amount by correcting the first rotation amount based on the second rotation amount in the calculation step (Step S402).

Each aspect of the present invention may include the following modified example. The optical sensor 60 (first sensor) determines a moving amount (insertion length) indicating the amount by which the insertion unit 2 moves in the longitudinal direction D1 of the insertion unit 2 when the insertion unit 2 is inserted into the subject.

Each aspect of the present invention may include the following modified example. The information-processing unit 40 (control unit) records insertion state information including the corrected rotation amount and the moving amount (insertion length) associated with each other on the memory 41 (recording medium).

Each aspect of the present invention may include the following modified example. The posture sensor 61 (second sensor) determines the posture of the sensor unit 6. The insertion state information includes posture information that is associated with the moving amount (insertion length) and indicates the posture of the sensor unit 6.

Each aspect of the present invention may include the following modified example. The insertion unit 2 includes the posture sensor 24 (third sensor) that is disposed in the distal end portion 2a including the distal end of the insertion unit 2 and determines the posture of the distal end portion 2a. The insertion state information includes posture information that is associated with the moving amount (insertion length) and indicates the posture of the distal end portion 2a.

Each aspect of the present invention may include the following modified example. The distal end portion 2a including the distal end of the insertion unit 2 is bendable inside a subject based on a bending instruction input through an operation of the operation unit 4. The insertion state information includes a bending amount that is associated with the moving amount (insertion length) and indicates the amount by which the distal end portion 2a has bent.

Each aspect of the present invention may include the following modified example. The insertion assistance unit 42 (control unit) generates operation information (insertion assistance information) indicating an operation required for inserting the insertion unit 2 into a subject by using a corrected rotation amount calculated in real time and the corrected rotation amount included in the insertion state information recorded on the memory 41 (recording medium).

Each aspect of the present invention may include the following modified example. The insertion assistance unit 42 (control unit) calculates the difference between a corrected rotation amount calculated in real time and the corrected rotation amount included in the insertion state information recorded on the memory 41 (recording medium) and generates operation information (insertion assistance information) by using the difference.

Each aspect of the present invention may include the following modified example. The insertion assistance unit 42 (control unit) calculates the corrected rotation amount by performing addition or subtraction using the first rotation amount and the second rotation amount.

In the first embodiment, the endoscope device 1 calculates a corrected rotation amount by correcting a relative rotation amount of the insertion unit 2 to the sensor unit 6 based on the rotation amount of the sensor unit 6. Therefore, the endoscope device 1 can accurately determine the rotation amount of the insertion unit 2.

The sensor unit 6 does not need to be fixed to a subject. As described above, a user may hold the sensor unit 6 by the hand. Even in such a case, the endoscope device 1 can accurately determine the rotation amount of the insertion unit 2.

The insertion state information is recorded on the memory 41. The insertion state information includes, for example, a corrected rotation amount of the insertion unit 2. The endoscope device 1 can record contents of the insertion operation in an inspection. A user can check whether the inspection has been performed along with a plan by referring to the insertion state information.

The insertion assistance unit 42 generates insertion assistance information by using information included in the insertion state information recorded on the memory 41 and outputs the generated insertion assistance information to the display unit 5. Even when a user is an unskilled worker, the user can easily perform the insertion operation in accordance with the insertion assistance information under various inspection conditions. The user easily enables the insertion unit 2 to reach an inspection portion.

### Second Embodiment

A second embodiment of the present invention will be described. A technique disclosed in Japanese Unexamined Patent Application, First Publication No. 2014-113352 described above does not provide a method of reproducing a reference position (rotation origin) of a rotation amount of an insertion unit in every inspection. Unless this rotation origin is fixed, it is difficult to calculate the rotation amount of the insertion unit. A holding unit and the insertion unit are integrated in this technique. Therefore, it is estimated that the holding unit and the insertion unit include a specific structure or a sensor. The structure fixes a relative position of the insertion unit to the holding unit. The sensor determines a positional relationship between the holding unit and the insertion unit with the holding unit and the insertion unit being close to each other.

It is preferable that the holding unit be detachable from the insertion unit. However, if the holding unit is detached from the insertion unit, it is difficult to use the above-described structure or sensor. As described above, many metal wires are woven in the surface of the insertion unit. The surface of the insertion unit has an even pattern formed by the metal wires. It is difficult to form a mark or the like indicating a rotation origin on the surface of the insertion unit.

In the first embodiment described above, when a skilled worker performs the equipment setting (operation O1), a reference image is recorded on the memory 41. When an unskilled worker performs the equipment setting (operation O3), the display unit 5 displays the reference image and displays a live image generated in real time by the imaging device 23. The unskilled worker adjusts the rotation amount of the insertion unit 2 such that the composition of the live image matches the composition of the reference image. In this way, a relative rotation position of the insertion unit 2 to a subject is adjusted.

However, a relative rotation position of the sensor unit 6 to the subject is not always adjusted through the above-described adjustment. Therefore, even when the composition of the live image matches the composition of the reference image, a relative rotation position (rotation origin) of the insertion unit 2 to the sensor unit 6 does not always match the rotation position in the composition of the reference image.

On the other hand, the second embodiment provides a method of adjusting the rotation origin of the insertion unit 2 without using an image. The posture sensor 61 and the posture sensor 24 determine a physical quantity that is based on the direction of gravity. In a case in which an inspection target is a pipe, an aircraft engine, or the like, the posture of the inspection target that is based on the direction of gravity hardly changes. Therefore, the endoscope device 1 can restrict a change of the rotation origin that is in accordance with a timing of an inspection by setting the rotation origin that is based on the direction of gravity.

The endoscope device 1 sets a positional relationship of rotation between the insertion unit 2 and the sensor unit 6 by using the value determined by each of the posture sensor 24 of the insertion unit 2 and the posture sensor 61 of the sensor unit 6. By doing this, the endoscope device 1 adjusts the rotation origin of the insertion unit 2 with respect to the sensor unit 6.

The posture sensor 24 may include only an acceleration sensor. The posture sensor 24 may determine a physical quantity that is based on the direction of a geomagnetic field. Accordingly, the posture sensor 24 may include only a geomagnetic sensor. The posture sensor 24 may include any two or three of an acceleration sensor, a gyro sensor, and a geomagnetic sensor. For example, the posture sensor 24 may include the acceleration sensor and the gyro sensor. Alternatively, the posture sensor 24 may include the acceleration sensor, the gyro sensor, and the geomagnetic sensor.

The posture sensor 61 may include only an acceleration sensor. The posture sensor 61 may determine a physical quantity that is based on the direction of the geomagnetic field. Accordingly, the posture sensor 61 may include only a geomagnetic sensor. The posture sensor 61 may include any two or three of an acceleration sensor, a gyro sensor, and a geomagnetic sensor. For example, the posture sensor 61 may include the acceleration sensor and the gyro sensor. Alternatively, the posture sensor 61 may include the acceleration sensor, the gyro sensor, and the geomagnetic sensor.

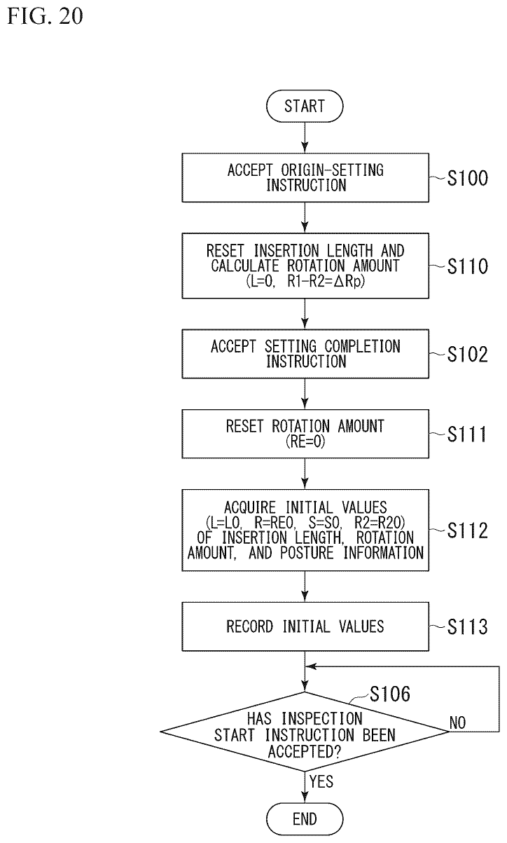

The state-recording processing shown in FIG. 11 is changed to state-recording processing shown in FIG. 20. FIG. 20 shows a procedure of the state-recording processing. The same processing as that shown in FIG. 11 will not be described.

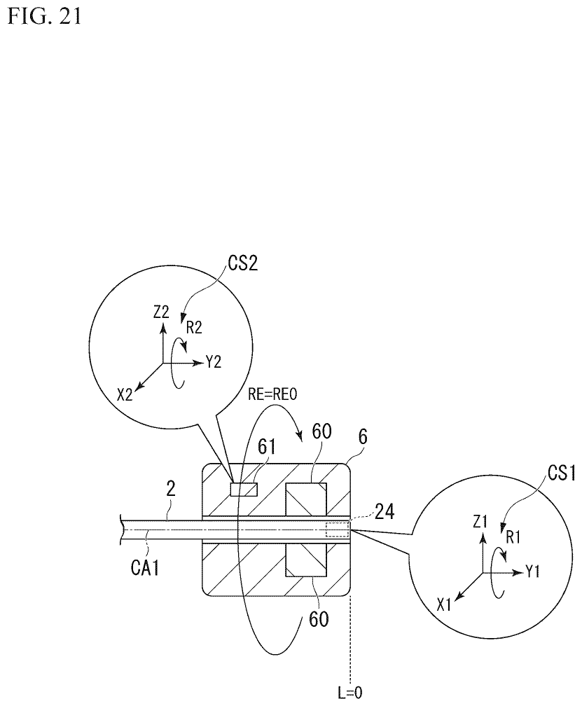

A skilled worker matches the position of the insertion unit 2 to the position of the sensor unit 6. FIG. 21 shows a positional relationship between the insertion unit 2 and the sensor unit 6 at this time. FIG. 21 shows a cross-section of the sensor unit 6. For example, the skilled worker matches the distal end surface of the insertion unit 2 to the end surface of the sensor unit 6. At this time, the skilled worker inputs an origin-setting instruction into the endoscope device 1 by operating the operation unit 4.

The operation-processing unit 33 outputs the origin-setting instruction to the state determination unit 34, the posture determination unit 35, and the information-processing unit 40 in Step S100. The state determination unit 34, the posture determination unit 35, and the information-processing unit 40 accept the origin-setting instruction in Step S100.

A coordinate system CS1 of the posture sensor 24 and a coordinate system CS2 of the posture sensor 61 are shown in FIG. 21. The coordinate system CS1 has an X1 axis, a Y1 axis, and a Z1 axis. The Y1 axis matches the center axis CA1 of the insertion unit 2. The coordinate system CS2 has an X2 axis, a Y2 axis, and a Z2 axis. The coordinate system CS1 and the coordinate system CS2 are set in advance such that the Y1 axis matches the Y2 axis when the distal end surface of the insertion unit 2 matches the end surface of the sensor unit 6. The X1 axis does not always match the X2 axis. The Z1 axis does not always match the Z2 axis.

After Step S100, the state determination unit 34 resets the insertion length calculated based on the value output from the optical sensor 60 to 0. The state determination unit 34, the posture determination unit 35, and the information-processing unit 40 execute processing related to a rotation amount (Step S110).

After the insertion length is reset to 0, a newly calculated insertion length indicates a moving amount of the insertion unit 2 in the longitudinal direction D1 of the insertion unit 2 after Step S110. Since the posture sensor 61 and the posture sensor 24 determine physical quantities that are based on the direction of gravity, the state determination unit 34 does not need to reset the posture calculated based on the value output from each of the posture sensor 61 and the posture sensor 24 to 0.

The state determination unit 34, the posture determination unit 35, and the information-processing unit 40 execute the following processing related to a rotation amount in Step S110.

The posture sensor 24 is capable of determining the direction of gravity. Accordingly, a relationship between the direction of gravity and the direction of the Y1 axis in the posture sensor 24 is known. The posture determination unit 35 calculates a rotation amount R1 of the insertion unit 2 around the Y1 axis based on the value output from the posture sensor 24.

The posture sensor 61 is capable of determining the direction of gravity. Accordingly, a relationship between the direction of gravity and the direction of the Y2 axis in the posture sensor 61 is known. The state determination unit 34 calculates a rotation amount R2 of the sensor unit 6 around the Y2 axis based on the value output from the posture sensor 61.

The information-processing unit 40 acquires the rotation amount R1 of the insertion unit 2 from the posture determination unit 35 and acquires the rotation amount R2 of the sensor unit 6 from the state determination unit 34. The information-processing unit 40 subtracts the rotation amount R2 of the sensor unit 6 from the rotation amount R1 of the insertion unit 2 so as to calculate a relative rotation amount (ΔRp). The relative rotation amount (ΔRp) indicates a positional relationship of rotation between the insertion unit 2 and the sensor unit 6. The information-processing unit 40 may subtract the rotation amount R1 of the insertion unit 2 from the rotation amount R2 of the sensor unit 6 so as to calculate the relative rotation amount (ΔRp). The information-processing unit 40 records the relative rotation amount (ΔRp) on the memory 41.

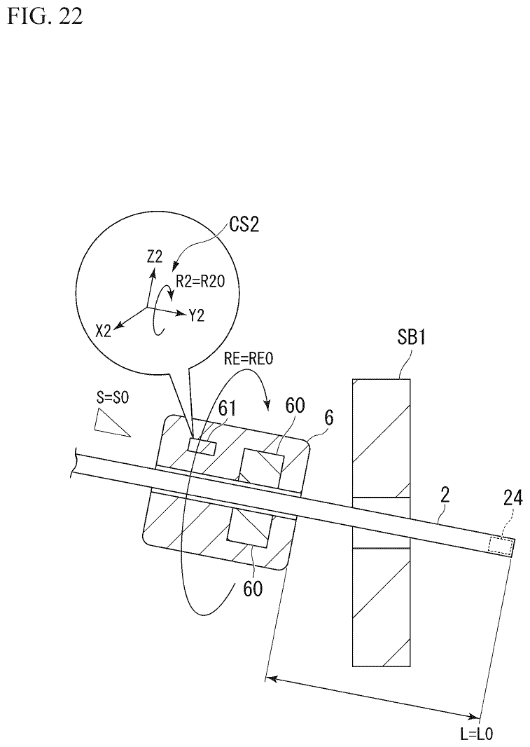

The skilled worker inserts the insertion unit 2 into a subject and sets a relative state of the insertion unit 2 to the subject to an initial state. FIG. 22 shows a positional relationship between a subject SB1 and the insertion unit 2 at this time. FIG. 22 shows cross-sections of the subject SB1 and the sensor unit 6.

The skilled worker adjusts the position of the insertion unit 2 so that the imaging device 23 can acquire an image of a portion in the subject SB1. In addition, the skilled worker adjusts the posture of the insertion unit 2 so that the insertion unit 2 can smoothly advance.

While the skilled worker is performing the above-described adjustment, the state determination unit 34 acquires a value determined by each of the optical sensor 60 and the posture sensor 61. The state determination unit 34 calculates an insertion length of the insertion unit 2 and a rotation amount R2 of the sensor unit 6. The state determination unit 34 calculates a posture of the sensor unit 6 and generates posture information of the sensor unit 6. While the skilled worker is performing the above-described adjustment, the posture determination unit 35 calculates a rotation amount R1 of the insertion unit 2.

After the state of the insertion unit 2 is set to an intended state, the skilled worker inputs a setting completion instruction into the endoscope device 1 by operating the operation unit 4. The operation-processing unit 33 outputs the setting completion instruction to the information-processing unit 40 in Step S102. The information-processing unit 40 accepts the setting completion instruction in Step S102.

After Step S102, the state determination unit 34 resets a rotation amount RE of the insertion unit 2 calculated based on the value output from the optical sensor 60 to 0 (Step S111). The rotation amount RE indicates a relative rotation amount of the insertion unit 2 to the sensor unit 6. The state determination unit 34 calculates a rotation amount RE. After the rotation amount RE is reset to 0, a newly calculated rotation amount RE indicates a rotation amount of the insertion unit 2 after Step S111.

After Step S111, the information-processing unit 40 acquires the insertion length of the insertion unit 2, the rotation amount RE of the insertion unit 2, the rotation amount R2 of the sensor unit 6, and the posture information of the sensor unit 6 from the state determination unit 34 (Step S112). For example, when the state of the insertion unit 2 is set to the state shown in FIG. 22, the insertion length of the insertion unit 2 is L0 and the rotation amount RE of the insertion unit 2 is RE0. In addition, the value indicating the posture (slope) of the sensor unit 6 is S0, and the rotation amount R2 of the sensor unit 6 is R20.

After Step S112, the information-processing unit 40 records the insertion length of the insertion unit 2, the rotation amount RE of the insertion unit 2, the rotation amount R2 of the sensor unit 6, and the posture information of the sensor unit 6 on the memory 41 (Step S113).

The skilled worker inputs an inspection start instruction into the endoscope device 1 by operating the operation unit 4 in order to start an inspection. The operation-processing unit 33 outputs the inspection start instruction to the state determination unit 34, the posture determination unit 35, and the information-processing unit 40 in Step S106. The state determination unit 34, the posture determination unit 35, and the information-processing unit 40 accept the inspection start instruction in Step S106. When the inspection start instruction has been accepted, the state-recording processing shown in FIG. 20 is completed.

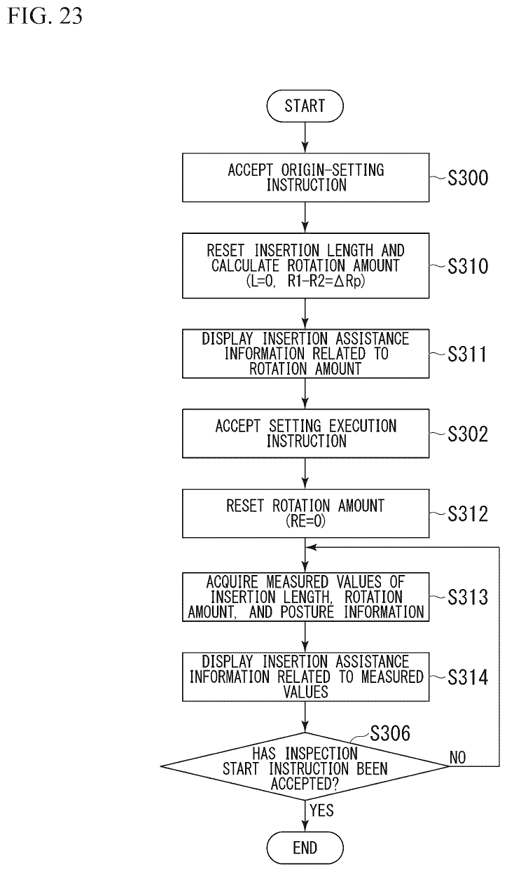

The equipment-setting processing shown in FIG. 16 is changed to equipment-setting processing shown in FIG. 23. FIG. 23 shows a procedure of the equipment-setting processing. The same processing as that shown in FIG. 16 will not be described.

The unskilled worker adjusts the position of the insertion unit 2 to the position of the sensor unit 6. At this time, the work performed by the unskilled worker is similar to that (FIG. 21) performed by the skilled worker. For example, the unskilled worker matches the distal end surface of the insertion unit 2 to the end surface of the sensor unit 6. At this time, the unskilled worker inputs an origin-setting instruction into the endoscope device 1 by operating the operation unit 4.

The operation-processing unit 33 outputs the origin-setting instruction to the state determination unit 34, the posture determination unit 35, and the information-processing unit 40 in Step S300. The state determination unit 34, the posture determination unit 35, and the information-processing unit 40 accept the origin-setting instruction in Step S300.

After Step S300, the state determination unit 34 resets the insertion length calculated based on the value output from the optical sensor 60 to 0. The state determination unit 34, the posture determination unit 35, and the information-processing unit 40 execute processing related to a rotation amount (Step S310).

After the insertion length is reset to 0, a newly calculated insertion length indicates a moving amount of the insertion unit 2 in the longitudinal direction D1 of the insertion unit 2 after Step S310. Since the posture sensor 61 and the posture sensor 24 determine physical quantities that are based on the direction of gravity, the state determination unit 34 does not need to reset the posture calculated based on the value output from each of the posture sensor 61 and the posture sensor 24 to 0.

The state determination unit 34, the posture determination unit 35, and the information-processing unit 40 execute the following processing related to a rotation amount in Step S310.

The posture determination unit 35 calculates a rotation amount R1 of the insertion unit 2 around the Y1 axis based on the value output from the posture sensor 24. The state determination unit 34 calculates a rotation amount R2 of the sensor unit 6 around the Y2 axis based on the value output from the posture sensor 61.

The information-processing unit 40 acquires the rotation amount R1 of the insertion unit 2 from the posture determination unit 35 and acquires the rotation amount R2 of the sensor unit 6 from the state determination unit 34. The information-processing unit 40 subtracts the rotation amount R2 of the sensor unit 6 from the rotation amount R1 of the insertion unit 2 so as to calculate a relative rotation amount (ΔRc). The relative rotation amount (ΔRc) indicates a positional relationship of rotation between the insertion unit 2 and the sensor unit 6. The information-processing unit 40 may subtract the rotation amount R1 of the insertion unit 2 from the rotation amount R2 of the sensor unit 6 so as to calculate the relative rotation amount (ΔRc).

The unskilled worker performs similar work to that performed by the skilled worker and realizes a similar state to that shown in FIG. 21. The unskilled worker sets the rotation state of the insertion unit 2 to the same state as that of the insertion unit 2 in the work performed by the skilled worker. The endoscope device 1 executes processing of assisting the work performed by the unskilled worker. Hereinafter, details of the processing will be described.

The insertion assistance unit 42 acquires the relative rotation amount (ΔRp) recorded on the memory 41 in Step S110. The insertion assistance unit 42 generates insertion assistance information related to the relative rotation amount (ΔRp) acquired from the memory 41 and the relative rotation amount (ΔRc) calculated in Step S310. The insertion assistance unit 42 outputs the insertion assistance information to the display unit 5 via the image-processing unit 30. The display unit 5 displays the insertion assistance information (Step S311).

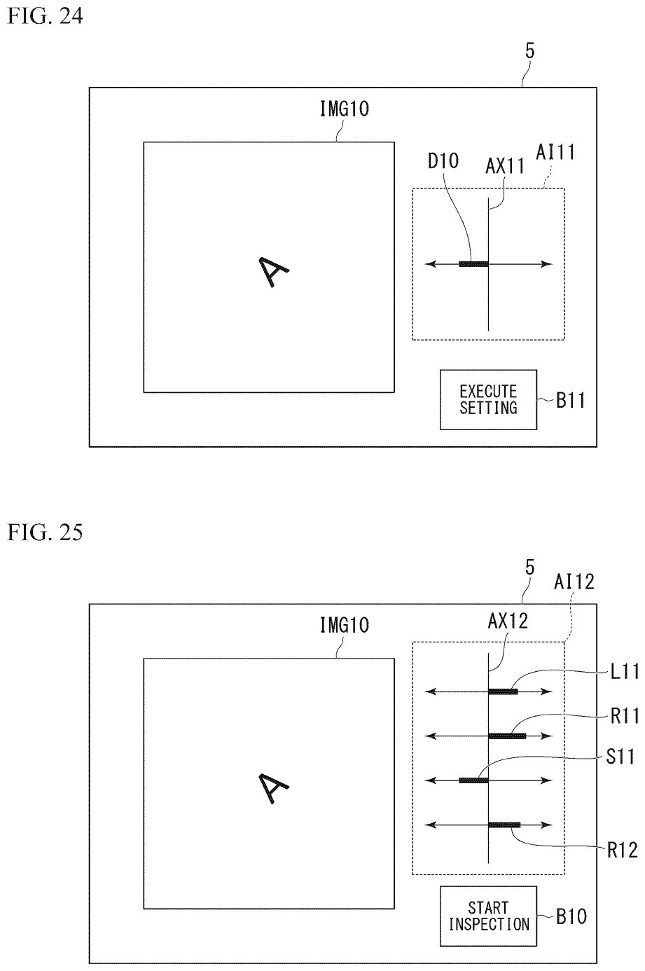

FIG. 24 shows information displayed on the display unit 5. The display unit 5 displays a live image IMG10, insertion assistance information AI11, and a button B11.

The live image IMG10 is a present image generated in real time by the imaging device 23.

The insertion assistance information AI11 includes difference information D10. The difference information D10 indicates the difference between the relative rotation amount (ΔRp) and the relative rotation amount (ΔRc). The relative rotation amount (ΔRp) is acquired from the memory 41 in Step S311. The relative rotation amount (ΔRc) is calculated in Step S310. The difference information D10 is displayed as a line having the length in accordance with the difference between the relative rotation amount (ΔRp) and the relative rotation amount (ΔRc). The difference information D10 is displayed on the right or left side of an axis AX11 in accordance with a relationship of the amount between the relative rotation amount (ΔRp) and the relative rotation amount (ΔRc).

The unskilled worker refers to the insertion assistance information AI11. The unskilled worker adjusts the rotation amount of the insertion unit 2 such that the difference corresponding to the difference information D10 matches 0. While the unskilled worker is adjusting the rotation amount of the insertion unit 2, the difference information D10 is updated in accordance with the operation performed by the unskilled worker.

When the above-described adjustment is completed, the unskilled worker inputs a setting execution instruction into the endoscope device 1 by operating the operation unit 4. For example, the unskilled worker presses the button B11 by operating the operation unit 4. By doing this, the unskilled worker can input the setting execution instruction into the endoscope device 1.

The operation-processing unit 33 outputs the setting execution instruction to the insertion assistance unit 42 in Step S302. The insertion assistance unit 42 accepts the setting execution instruction in Step S302.

The state determination unit 34 acquires the value determined by each of the optical sensor 60 and the posture sensor 61. The state determination unit 34 calculates an insertion length of the insertion unit 2 and a rotation amount R2 of the sensor unit 6. The state determination unit 34 calculates a posture of the sensor unit 6 and generates posture information of the sensor unit 6.

After Step S302, the state determination unit 34 resets a rotation amount RE of the insertion unit 2 calculated based on the value output from the optical sensor 60 to 0 (Step S312). The rotation amount RE indicates a relative rotation amount of the insertion unit 2 to the sensor unit 6. The state determination unit 34 calculates a rotation amount RE. After the rotation amount RE is reset to 0, a newly calculated rotation amount RE indicates a rotation amount of the insertion unit 2 after Step S312.

After Step S312, the insertion assistance unit 42 acquires the insertion length of the insertion unit 2, the rotation amount RE of the insertion unit 2, the rotation amount R2 of the sensor unit 6, and the posture information of the sensor unit 6 from the state determination unit 34 (Step S313).

After Step S313, the insertion assistance unit 42 acquires the information recorded on the memory 41 in Step S113. In other words, the insertion assistance unit 42 acquires the insertion length (L0) of the insertion unit 2, the rotation amount RE (RE0) of the insertion unit 2, the rotation amount R2 (R20) of the sensor unit 6, and the posture information (S0) of the sensor unit 6. The insertion assistance unit 42 generates insertion assistance information by using the information acquired from the memory 41 and the information acquired from the state determination unit 34 in Step S313. The insertion assistance unit 42 outputs the insertion assistance information to the display unit 5 via the image-processing unit 30. The display unit 5 displays the insertion assistance information (Step S314).

FIG. 25 shows information displayed on the display unit 5. The display unit 5 displays a live image IMG10, insertion assistance information AI12, and a button B10.

The live image IMG10 is a present image generated in real time by the imaging device 23.

The insertion assistance information AI12 includes insertion length information L11. The insertion length information L11 indicates the difference between the previous insertion length (L0) and the present insertion length. The previous insertion length (L0) is acquired from the memory 41 in Step S314. The present insertion length is acquired from the state determination unit 34 in Step S313. The insertion length information L11 is displayed as a line having the length in accordance with the amount of the difference. The insertion length information L11 is displayed on the right or left side of an axis AX12 in accordance with a relationship of the amount between the previous insertion length (L0) and the present insertion length.

The insertion assistance information AI12 includes rotation amount information R11. The rotation amount information R11 indicates the difference between the previous rotation amount RE (RE0) and the present rotation amount RE. The previous rotation amount RE (RE0) is acquired from the memory 41 in Step S314. The present rotation amount RE is acquired from the state determination unit 34 in Step S313. The rotation amount information R11 is displayed as a line having the length in accordance with the amount of the difference. The rotation amount information R11 is displayed on the right or left side of the axis AX12 in accordance with a relationship of the amount between the previous rotation amount RE (RE0) and the present rotation amount RE.

The insertion assistance information AI12 includes posture information S11. The posture information S11 indicates the difference between the previous value (S0) of the posture information of the sensor unit 6 and the present value of the posture information of the sensor unit 6. The previous value (S0) of the posture information is acquired from the memory 41 in Step S314. The present value of the posture information is acquired from the state determination unit 34 in Step S313. The posture information S11 is displayed as a line having the length in accordance with the amount of the difference. The posture information S11 is displayed on the right or left side of the axis AX12 in accordance with a relationship of the amount between the previous value (S0) of the posture information and the present value of the posture information. The posture information S11 indicates the posture of the insertion unit 2 in the hole H1.

The insertion assistance information AI12 includes rotation amount information R12. The rotation amount information R12 indicates the difference between the previous rotation amount R2 (R20) of the sensor unit 6 and the present rotation amount R2 of the sensor unit 6. The previous rotation amount R2 (R20) is acquired from the memory 41 in Step S314. The present rotation amount R2 is acquired from the state determination unit 34 in Step S313. The rotation amount information R12 is displayed as a line having the length in accordance with the amount of the difference. The rotation amount information R12 is displayed on the right or left side of the axis AX12 in accordance with a relationship of the amount between the previous rotation amount R2 (R20) and the present rotation amount R2.

The unskilled worker refers to the insertion assistance information AI12. The unskilled worker adjusts the position of the insertion unit 2 such that the difference corresponding to the insertion length information L11 matches 0. The unskilled worker adjusts the rotation amount of the insertion unit 2 such that the difference corresponding to the rotation amount information R11 matches 0. The unskilled worker adjusts the posture of the insertion unit 2 such that the difference corresponding to the posture information S11 matches 0. The unskilled worker adjusts the rotation amount of the sensor unit 6 such that the difference corresponding to the rotation amount information R12 matches 0. The insertion length information L11, the rotation amount information R11, the posture information S11, and the rotation amount information R12 are updated in accordance with the operation performed by the unskilled worker while the unskilled worker is adjusting the position, the rotation amount, and the posture of the insertion unit 2 and is adjusting the rotation amount of the sensor unit 6.

When the difference corresponding to the rotation amount information R11 matches 0, the relative rotation amount (ΔRc) is the same as the relative rotation amount (ΔRp). After the rotation amount RE of the insertion unit 2 is reset to 0 in Step S312, the unskilled worker adjusts the rotation amount of the insertion unit 2 such that the present rotation amount RE matches the previous rotation amount RE (RE0). When the present rotation amount RE matches the previous rotation amount RE (RE0), the reference position of rotation amounts of the insertion unit 2 and the sensor unit 6 around the center axis CA1 of the insertion unit 2 is set to the same as a previous reference position. In other words, a rotation origin of the insertion unit 2 with respect to the sensor unit 6 is set to the same as a previous rotation origin. At this time, the present positional relationship of rotation between the insertion unit 2 and the sensor unit 6 matches a positional relationship of rotation between the insertion unit 2 and the sensor unit 6 in a previous inspection performed by the skilled worker.

When the difference corresponding to the rotation amount information R12 matches 0, the present rotation amount R2 of the sensor unit 6 is the same as the rotation amount R2 (R20) of the sensor unit 6 in a previous inspection. At this time, the present rotation state of the sensor unit 6 matches a rotation state of the sensor unit 6 in the previous inspection. Due to this, the unskilled worker can reproduce movement of the hand of the skilled worker.

When the above-described adjustment is completed, the unskilled worker inputs an inspection start instruction into the endoscope device 1 by operating the operation unit 4 in order to start an inspection. For example, the unskilled worker presses the button B10 by operating the operation unit 4. By doing this, the unskilled worker can input the inspection start instruction into the endoscope device 1.

The operation-processing unit 33 outputs the inspection start instruction to the state determination unit 34, the posture determination unit 35, and the insertion assistance unit 42 in Step S306. The state determination unit 34, the posture determination unit 35, and the insertion assistance unit 42 accept the inspection start instruction in Step S306. When the inspection start instruction has been accepted, the equipment-setting processing shown in FIG. 23 is completed. Step S313 and Step S314 are repeated until the inspection start instruction is accepted.

The insertion assistance unit 42 may display a three-dimensional model of the insertion unit 2 on the display unit 5. The insertion assistance unit 42 may display a target of the rotation amount of the insertion unit 2 and a target of the posture of the insertion unit 2 on the three-dimensional model. Due to this, visibility of information required for adjusting the rotation amount and the posture of the insertion unit 2 is improved.

A procedure of history-recording processing in the second embodiment is the same as that shown in FIG. 14. A procedure of insertion assistance processing in the second embodiment is the same as that shown in FIG. 18.

The information-processing unit 40 may record the rotation amount R2 of the sensor unit 6 on the memory 41 in Step S203 in the history-recording processing in the first embodiment or the second embodiment. Due to this, movement of the hand of the skilled worker who is holding the sensor unit 6 is recorded. The insertion assistance unit 42 may generate insertion assistance information related to the rotation amount R2 of the sensor unit 6 recorded on the memory 41 and the rotation amount R2 of the sensor unit 6 acquired from the state determination unit 34 in Step S401 in the insertion assistance processing in the first embodiment or the second embodiment. The insertion assistance unit 42 may display the insertion assistance information on the display unit 5 in the insertion assistance processing in the first embodiment or the second embodiment. The unskilled worker can become aware of a timing at which the skilled worker twists the sensor unit 6.

Each aspect of the present invention may include the following modified example. The information-processing unit 40 (control unit) records insertion state information including a second rotation amount (rotation amount R2) and a moving amount (insertion length) associated with each other on the memory 41 (recording medium).

Each aspect of the present invention may include the following modified example. The insertion unit 2 includes the posture sensor 24 (third sensor) that is disposed in the distal end portion 2a including the distal end of the insertion unit 2 and determines a third rotation amount (rotation amount R1) indicating a rotation amount of the insertion unit 2 around the center axis CA1 of the insertion unit 2. The information-processing unit 40 resets a relative rotation amount of the insertion unit 2 to the sensor unit 6 by using the second rotation amount and the third rotation amount.

In the second embodiment, the endoscope device 1 can adjust a rotation origin of the insertion unit 2 with respect to the sensor unit 6 by using the rotation amount R1 of the insertion unit 2 and the rotation amount R2 of the sensor unit 6. The endoscope device 1 does not need to use an image generated by the imaging device 23 in order to adjust the rotation origin. Therefore, the number of elements visually checked by a user (inspector) reduce, and accuracy and efficiency of adjustment of the rotation origin is improved.

When the operation unit 4 is fixed to the sensor unit 6 as shown in FIG. 8, a structure including the operation unit 4 and the sensor unit 6 does not have rotational symmetry with respect to the center axis CA1 of the insertion unit 2. In addition, when the insertion unit 2 tends to easily bend in a certain direction, the insertion unit 2 does not have rotational symmetry with respect to the center axis CA1. In order to skilledly insert the insertion unit 2 into a subject, a user needs to perform the bending operation and rotate the insertion unit 2 in view of a rotational tendency of the insertion unit 2.

The unskilled worker needs to match a positional relationship of rotation between the insertion unit 2 and the sensor unit 6 to a positional relationship in a previous inspection performed by the skilled worker. Even when the unskilled worker rotates the insertion unit 2 like the skilled worker does in a state in which the positional relationship in a present inspection does not match the positional relationship in the previous inspection, the unskilled worker may have difficulties in skilledly inserting the insertion unit 2 into a subject.

In the second embodiment, the unskilled worker adjusts a rotation origin of the insertion unit 2 with respect to the sensor unit 6. In addition, the unskilled worker adjusts rotation amounts of the insertion unit 2 and the sensor unit 6 such that the rotation amount R2 of the sensor unit 6 in the present inspection matches the rotation amount R2 of the sensor unit 6 in a previous inspection. Due to this, it is highly probable that the unskilled worker skilledly inserts the insertion unit 2 into a subject.

### Third Embodiment

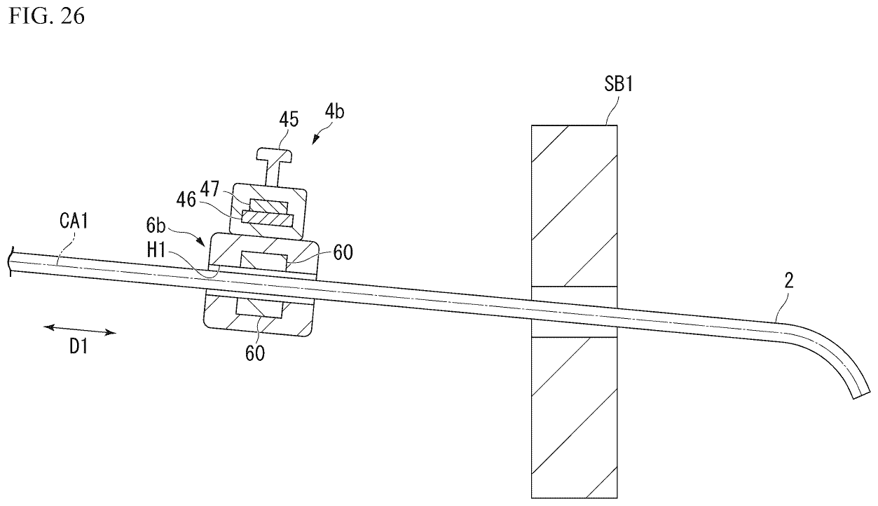

A third embodiment of the present invention will be described. The operation unit 4 shown in FIG. 1 and the like is changed to an operation unit 4b shown in FIG. 26. The sensor unit 6 shown in FIG. 1 and the like is changed to a sensor unit 6b shown in FIG. 26. FIG. 26 shows cross-sections of the operation unit 4b and the sensor unit 6b.

The operation unit 4b is fixed to the sensor unit 6b. The operation unit 4b includes a joystick 45, a substrate 46, and a posture sensor 47. The joystick 45 is the same as the joystick 45 shown in FIG. 8. The substrate 46 is the same as the substrate 46 shown in FIG. 8. The posture sensor 47 is the same as the posture sensor 61 shown in FIG. 5 and the like. The posture sensor 47 is disposed inside the operation unit 4b and is fixed to the operation unit 4b.

The posture sensor 47 is disposed on the substrate 46. A value determined by the posture sensor 47 is output to the operation-processing unit 33 via the substrate 46. The posture sensor 47 may generate a bending instruction in accordance with the posture or movement of the operation unit 4b. The operation-processing unit 33 may output the bending instruction generated by the posture sensor 47 to the bending control unit 39.

The sensor unit 6b includes an optical sensor 60. The sensor unit 6b does not include the posture sensor 61 shown in FIG. 5 and the like.

A hole H1 through which the insertion unit 2 passes is formed in the sensor unit 6b. The insertion unit 2 can move in a longitudinal direction D1 of the insertion unit 2 in the hole H1. In addition, the insertion unit 2 can rotate around a center axis CA1 of the insertion unit 2 in the hole H1.

The operation-processing unit 33 acquires the value determined by the posture sensor 47. The operation-processing unit 33 calculates a posture of the operation unit 4b and generates posture information of the operation unit 4b. Since the operation unit 4b is fixed to the sensor unit 6b, the posture information of the operation unit 4b indicates the posture of the sensor unit 6b. Since the insertion unit 2 passes through the hole H1 formed in the sensor unit 6b, the posture of the sensor unit 6b is the same as that of the insertion unit 2 in the hole H1. Therefore, the posture information of the operation unit 4b indicates the posture of the insertion unit 2 in the hole H1.

The operation unit 4b may be attachable to and detachable from the sensor unit 6b. The operation unit 4b may include a sensor that determines a state of connection between the operation unit 4b and the sensor unit 6b. The substrate 46 may include a control circuit that determines the state of the connection between the operation unit 4b and the sensor unit 6b based on a value output from the sensor.

The control circuit may output the value determined by the posture sensor 47 to the operation-processing unit 33 only when the operation unit 4b is attached to the sensor unit 6b. Alternatively, the control circuit may output information indicting the state of the connection between the operation unit 4b and the sensor unit 6b to the operation-processing unit 33. The operation-processing unit 33 may determine the state of the connection between the operation unit 4b and the sensor unit 6b by using the information. The operation-processing unit 33 may determine that the value determined by the posture sensor 47 is effective and may process the value only when the operation unit 4b is attached to the sensor unit 6b.

Each aspect of the present invention may include the following modified example. The distal end portion 2a including the distal end of the insertion unit 2 is bendable inside a subject based on a bending instruction input through an operation of the operation unit 4b. The posture sensor 47 (second sensor) is disposed in the operation unit 4b.

Each aspect of the present invention may include the following modified example. The operation unit 4b is attachable to and detachable from the sensor unit 6b. When the operation unit 4b is attached to the sensor unit 6b, the posture sensor 47 (second sensor) determines a second rotation amount of the sensor unit 6b.

In the third embodiment, the sensor unit 6b does not include the posture sensor 61, and the operation unit 4b includes the posture sensor 47. The sensor unit 6b is more miniaturized than the sensor unit 6.

### Fourth Embodiment

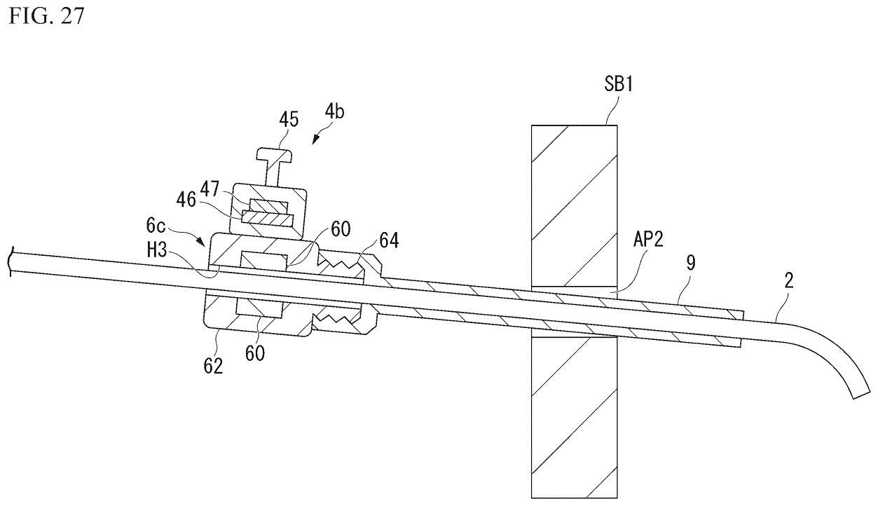

A fourth embodiment of the present invention will be described. The operation unit 4 shown in FIG. 1 and the like is changed to an operation unit 4b shown in FIG. 27. The sensor unit 6 shown in FIG. 1 and the like is changed to a sensor unit 6c shown in FIG. 27. FIG. 27 shows cross-sections of the operation unit 4b and the sensor unit 6c. The operation unit 4b is the same as the operation unit 4b shown in FIG. 26.

The sensor unit 6c includes a main body unit 62 and a screw part 64. The main body unit 62 includes the optical sensor 60. The screw part 64 is connected to the main body unit 62. A male screw is formed on the surface of the screw part 64. A hole H3 through which the insertion unit 2 passes is formed in the main body unit 62 and the screw part 64.

The sensor unit 6c is connected to a guide tube 9. The guide tube 9 is a tubular auxiliary component. A hole through which the insertion unit 2 passes is formed in the guide tube 9. The male screw of the screw part 64 fits a female screw of the guide tube 9, and the sensor unit 6c is fixed to the guide tube 9.

The insertion unit 2 is inserted into a subject SB1. An access port AP2 is formed in the subject SB1. The guide tube 9 is inserted into the subject SB1 through the access port AP2.

In a case in which the structure of the subject SB1 near the access port AP2 is complicated, the guide tube 9 is used to restrict the position of the distal end of the insertion unit 2 beforehand. The guide tube 9 can maintain the posture of the insertion unit 2.

In the fourth embodiment, the operation unit 4b, the sensor unit 6c, and the guide tube 9 are fixed to each other. Since the posture of the insertion unit 2 is easily maintained, operability is improved.

In a case in which the distance between the sensor unit 6c and the subject SB1 changes, a change of the distance may be accidentally determined as a moving amount of the insertion unit 2 and the insertion length may contain an error. By using the guide tube 9, the distance between the sensor unit 6c and the subject SB1 is likely to be fixed.

A graduation may be displayed on the side of the guide tube 9. A user may refer to the graduation and may maintain the distance between the sensor unit 6c and the subject SB1.

### Fifth Embodiment

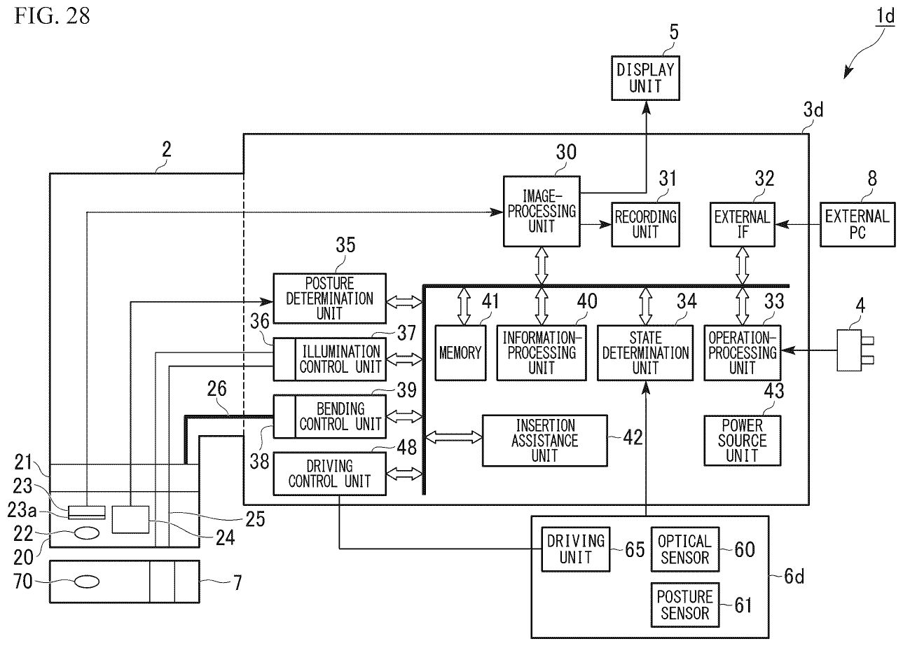

A fifth embodiment of the present invention will be described. The endoscope device 1 shown in FIG. 4 is changed to an endoscope device 1d shown in FIG. 28. FIG. 28 shows an internal configuration of the endoscope device 1d. The same configuration as that shown in FIG. 4 will not be described.

The main body unit 3 shown in FIG. 4 is changed to a main body unit 3d shown in FIG. 28. The main body unit 3d includes an image-processing unit 30, a recording unit 31, an external interface (IF) 32, an operation-processing unit 33, a state determination unit 34, a posture determination unit 35, a light source 36, an illumination control unit 37, a motor 38, a bending control unit 39, an information-processing unit 40, a memory 41, an insertion assistance unit 42, a power source unit 43, and a driving control unit 48.

The sensor unit 6 shown in FIG. 4 is changed to a sensor unit 6d. The sensor unit 6d includes an optical sensor 60, a posture sensor 61, and a driving unit 65. The optical sensor 60 is the same as the optical sensor 60 shown in FIG. 4. The posture sensor 61 is the same as the posture sensor 61 shown in FIG. 4.

The driving unit 65 includes a motor, a gear, and a roller. The roller is in contact with the side of the insertion unit 2. The driving unit 65 drives the roller by using the motor and the gear. A friction force occurs between the roller and the insertion unit 2. The insertion unit 2 moves in the longitudinal direction D1 of the insertion unit 2 or rotates around the center axis CA1 of the insertion unit 2 in accordance with the friction force. The driving control unit 48 outputs a driving signal to the driving unit 65 and controls the driving unit 65.

There is a case in which the roller slips on the surface of the insertion unit 2. In such a case, a rotation amount of the roller is not correctly determined. Therefore, it is difficult to correctly determine a rotation amount of the insertion unit 2 by using the rotation amount of the roller.

In the fifth embodiment, the endoscope device 1d includes the optical sensor 60 that determines a rotation amount of the insertion unit 2 without touching the insertion unit 2. Therefore, the endoscope device 1d can accurately determine the rotation amount of the insertion unit 2.

While preferred embodiments of the invention have been described and shown above, it should be understood that these are examples of the invention and are not to be considered as limiting. Additions, omissions, substitutions, and other modifications can be made without departing from the spirit or scope of the present invention. Accordingly, the invention is not to be considered as being limited by the foregoing description, and is only limited by the scope of the appended claims.

What is claimed is:

1. An insertion state determination method executed by a processor, the method comprising:
acquiring a first rotation amount when an elongated insertion unit of an endoscope device is inserted into a subject, wherein the first rotation amount indicates a rotation amount of the insertion unit around a center axis of the insertion unit and is determined by a first sensor disposed in a sensor unit in which a hole through which the insertion unit passes is formed;
acquiring a second rotation amount when the insertion unit is inserted into the subject, wherein the second rotation amount indicates a rotation amount of the sensor unit around the center axis and is determined by a second sensor disposed in the sensor unit or an object fixed to the sensor unit; and
calculating a corrected rotation amount by correcting the first rotation amount based on the second rotation amount.

2. The insertion state determination method according to claim 1, wherein the first sensor is configured to determine a moving amount indicating an amount by which the insertion unit moves in a longitudinal direction of the insertion unit when the insertion unit is inserted into the subject.

3. The insertion state determination method according to claim 2, further comprising recording insertion state information including the corrected rotation amount and the moving amount associated with each other on a recording medium.

4. The insertion state determination method according to claim 2, further comprising recording insertion state information including the second rotation amount and the moving amount associated with each other on a recording medium.

5. The insertion state determination method according to claim 3, wherein
the second sensor is configured to determine a posture of the sensor unit, and
the insertion state information further includes posture information that is associated with the moving amount and indicates the posture.

6. The insertion state determination method according to claim 4, wherein
the second sensor is configured to determine a posture of the sensor unit, and
the insertion state information further includes posture information that is associated with the moving amount and indicates the posture.

7. The insertion state determination method according to claim 3, wherein
the insertion unit includes a third sensor that is disposed in a distal end portion including a distal end of the insertion unit and is configured to determine a posture of the distal end portion, and
the insertion state information further includes posture information that is associated with the moving amount and indicates the posture.

8. The insertion state determination method according to claim 4, wherein
the insertion unit includes a third sensor that is disposed in a distal end portion including a distal end of the insertion unit and is configured to determine a posture of the distal end portion, and
the insertion state information further includes posture information that is associated with the moving amount and indicates the posture.

9. The insertion state determination method according to claim 3, wherein
a distal end portion including a distal end of the insertion unit is bendable inside the subject based on a bending instruction input through an input device that accepts an operation performed by a user, and
the insertion state information further includes a bending amount that is associated with the moving amount and indicates an amount by which the distal end portion has bent.

10. The insertion state determination method according to claim 4, wherein
a distal end portion including a distal end of the insertion unit is bendable inside the subject based on a bending instruction input through an input device that accepts an operation performed by a user, and
the insertion state information further includes a bending amount that is associated with the moving amount and indicates an amount by which the distal end portion has bent.

11. The insertion state determination method according to claim 3, further comprising generating operation information indicating an operation required for inserting the insertion unit into the subject by using the corrected rotation amount calculated in real time and the corrected rotation amount included in the insertion state information recorded on the recording medium.

12. The insertion state determination method according to claim 4, further comprising generating operation information indicating an operation required for inserting the insertion unit into the subject by using the corrected rotation amount calculated in real time and the corrected rotation amount included in the insertion state information recorded on the recording medium.

13. The insertion state determination method according to claim 11, wherein generating the operation information comprises:
calculating a difference between the corrected rotation amount calculated in real time and the corrected rotation amount included in the insertion state information recorded on the recording medium; and
generating the operation information by using the difference.

14. The insertion state determination method according to claim 12, wherein generating the operation information comprises:
calculating a difference between the corrected rotation amount calculated in real time and the corrected rotation amount included in the insertion state information recorded on the recording medium; and
generating the operation information by using the difference.

15. The insertion state determination method according to claim 1, wherein
the insertion unit includes a third sensor that is disposed in a distal end portion including a distal end of the insertion unit and is configured to determine a third rotation amount indicating a rotation amount of the insertion unit around a center axis of the insertion unit, and
the method further comprises resetting a relative rotation amount of the insertion unit to the sensor unit by using the second rotation amount and the third rotation amount.

16. The insertion state determination method according to claim 1, wherein
a distal end portion including a distal end of the insertion unit is bendable inside the subject based on a bending instruction input through an input device that accepts an operation performed by a user, and
the second sensor is disposed in the input device.

17. The insertion state determination method according to claim 16, wherein
the input device is attachable to and detachable from the sensor unit, and
when the input device is attached to the sensor unit, the second sensor is configured to determine the second rotation amount.

18. The insertion state determination method according to claim 1, further comprising calculating the corrected rotation amount comprises calculating the corrected rotation amount by performing addition or subtraction using the first rotation amount and the second rotation amount.
